(19) United States
(12) Patent Application Publication
(10) Pub. No.: US 2026/0034365 A1
(43) Pub. Date: Feb. 5, 2026

(54) ASSISTED PROGRAMMING SYSTEM FOR NEURAL STIMULATION THERAPY

(71) Applicant: Saluda Medical Pty Limited, Macquarie Park (AU)

(72) Inventors: Daniel John Parker, Macquarie Park (AU); Peter Minarik, Macquarie Park (AU); Stella H. Kim, Minneapolis, MN (US); Theoderick Tam, San Jose, CA (US)

(21) Appl. No.: 19/288,312

(22) Filed: Aug. 1, 2025

Related U.S. Application Data
(60) Provisional application No. 63/678,153, filed on Aug. 1, 2024.

(51) Int. Cl. A61N 1/36; A61N 1/372

(52) U.S. Cl. CPC A61N 1/36146; A61N 1/37247

(57) ABSTRACT

Disclosed is a neurostimulation system and method. A control unit controls a stimulus source to deliver neural stimuli via selected electrodes to a neural pathway according to a stimulus intensity parameter. An external computing device in communication with the neurostimulation device comprises a display and processor. The processor initialises the stimulus intensity parameter, and renders on the display a stimulation control and a graphical element adjacent the stimulation control. Upon user activation of the stimulation control, the processor ramps a value of the stimulus intensity parameter, while instructing the control unit to cause delivery of stimuli according to the ramping value of the stimulus intensity parameter. Upon user deactivation of the stimulation control, the processor ceases ramping the value of the stimulus intensity parameter. The graphical element is configured to dynamically indicate the ramping value of the stimulus intensity parameter; and indicate a discomfort threshold for the selected electrodes.

### CROSS-REFERENCE TO RELATED APPLICATIONS

The present application claims priority from U.S. Provisional Patent Application No. 63/678,153 filed on 1 Aug. 2024, the contents of which are incorporated herein by reference in their entirety.

### TECHNICAL FIELD

The present invention relates to neural stimulation therapy and in particular to improved methods of programming closed-loop neural stimulation devices and systems.

### BACKGROUND OF THE INVENTION

There are a range of situations in which it is desirable to apply neural stimuli in order to alter neural function, a process known as neuromodulation. For example, neuromodulation is used to treat a variety of disorders including chronic pain, movement disorders, and voiding disorders. A neuromodulation device applies an electrical pulse (stimulus) to neural tissue (fibres, or neurons) in order to generate a therapeutic effect. In general, the electrical stimulus generated by a neuromodulation device evokes a neural response known as an action potential in a neural fibre which then has either inhibitory or excitatory effects on neural networks. Inhibitory effects can be used to modulate an undesired process such as the transmission of pain, or excitatory effects may be used to cause a desired effect such as the contraction of a muscle.

When used to relieve neuropathic pain originating in the trunk and limbs, the electrical pulse is applied to the dorsal column (DC) of the spinal cord, a procedure referred to as spinal cord stimulation (SCS). Such a device typically comprises an implanted electrical pulse generator, and a power source such as a battery that may be transcutaneously rechargeable by wireless means, such as inductive transfer. An electrode array is connected to the pulse generator, and is implanted adjacent the target neural fibre(s) in the spinal cord, typically in the dorsal epidural space above the dorsal column. An electrical pulse of sufficient intensity applied to the target neural fibres by a stimulus electrode causes the depolarisation of neurons in the fibres, which in turn generates an action potential in the fibres. Action potentials propagate along the fibres in an orthodromic direction (in afferent fibres this means towards the head, or rostral) and in an antidromic direction (in afferent fibres this means towards the cauda, or caudal). Action potentials propagating along Aβ (A-beta) fibres being stimulated in this way may inhibit the transmission of pain from a region of the body innervated by the target neural fibres (the dermatome) to the brain. To sustain the pain relief effects, stimuli are applied repeatedly, for example at a stimulus frequency in the range of 30 Hz-100 Hz.

For effective and comfortable neuromodulation, it is necessary to maintain stimulus intensity above a recruitment threshold. Stimuli below the recruitment threshold will fail to recruit sufficient neurons to generate action potentials with a therapeutic effect. In some neuromodulation applications, response from a single class of fibre is desired, but the stimulus waveforms employed can evoke action potentials in other classes of fibres which cause unwanted side effects. In pain relief, it is therefore desirable to apply stimuli with intensity below a discomfort threshold, above which uncomfortable or painful percepts arise due to over-recruitment of Aβ fibres or recruitment of undesired fibre classes. When recruitment is too large, Aβ fibres produce uncomfortable sensations. Stimulation at high intensity may even recruit Aδ (A-delta) fibres, which are sensory nerve fibres associated with acute pain, cold and heat sensation. It is therefore desirable to maintain stimulus intensity within a therapeutic range between the recruitment threshold and the discomfort threshold.

The task of maintaining appropriate neural recruitment is made more difficult by electrode migration (change in position over time) or postural changes of the implant recipient (patient), either of which can significantly alter the neural recruitment arising from a given stimulus, and therefore the therapeutic range. The spinal cord itself moves within the cerebrospinal fluid (CSF) with respect to the dura and the electrode array. During postural changes, the distance between the spinal cord and the electrode can change significantly. This effect is so large that postural changes alone can cause a previously comfortable and effective stimulus regime to become either ineffectual or painful.

Attempts have been made to address such problems by way of feedback or closed-loop control, such as using the methods set forth in International Patent Publication No. WO2012/155188 by the present applicant, the content of which is incorporated herein by reference. Feedback control seeks to compensate for relative nerve/electrode movement by controlling the intensity of the delivered stimuli to maintain neural recruitment at or near a target value. The intensity of a neural response evoked by a stimulus may be used as a feedback variable representative of the amount of neural recruitment. A signal representative of the neural response may be sensed by a measurement electrode in electrical communication with the recruited neural fibres, and processed to obtain the feedback variable. Based on the response intensity, the intensity of the applied stimulus may be adjusted to bring the response intensity closer to the target value.

It is therefore desirable to accurately measure the intensity and other characteristics of a neural response evoked by the stimulus. The action potentials generated by the depolarisation of a large number of fibres by a stimulus sum to form a measurable signal known as an evoked compound action potential (ECAP). Accordingly, an ECAP is the sum of responses from a large number of single fibre action potentials. The ECAP generated from the depolarisation of a group of similar fibres may be sensed by a measurement electrode as a positive peak potential, then a negative peak, followed by a second positive peak. This morphology is caused by the region of activation passing the measurement electrode as the action potentials propagate along the individual fibres.

Approaches proposed for obtaining a neural response measurement are described by the present applicant in International Patent Publication No. WO2012/155183, the content of which is incorporated herein by reference.

Closed-loop neural stimulation therapy is governed by a number of parameters to which values must be assigned to implement the therapy. The effectiveness of the therapy depends in large measure on the suitability of the assigned parameter values to the patient undergoing the therapy. As patients vary significantly in their physiological characteristics, a “one-size-fits-all” approach to parameter value assignment is likely to result in ineffective therapy for a large proportion of patients. An important preliminary task, once a neuromodulation device has been implanted in a patient, is therefore to assign values to the therapy parameters that maximise the effectiveness of the therapy the device will deliver to that particular patient. This task is known as programming or fitting the device. Programming generally involves applying certain test stimuli via the device, recording responses, and based on the recorded responses, inferring or calculating the most effective parameter values for the patient. The resulting parameter values are then formed into a “program” that may be loaded to the device to govern subsequent therapy. Some of the recorded responses may be neural responses evoked by the test stimuli, which provide an objective source of information that may be analysed along with subjective responses elicited from the patient. In an effective programming system, the more responses that are analysed, the more effective the eventual assigned parameter values should be.

However, programming may be costly and time-consuming if unnecessarily prolonged. There is therefore an incentive to minimise the number of test stimuli to be applied and the amount of information to be recorded and analysed in order to produce the assigned values of the therapy parameters. In particular, the size of the therapy parameter search space is such that testing every possible combination of therapy parameters is impractical.

Moreover, programming workflows are generally conducted by a trained clinician or engineer who mediates between the patient and the programming system by interpreting the patient's subjective verbal responses. However, this mediation may be problematic, particularly when patients lack the capacity to express the sensations they are feeling during the test stimuli. In addition, the subjective responses of the patient, even if clearly expressed, are not always a reliable guide to the device's effect on the patient. This can lead to inefficient programming and, in a worst case, ineffective assigned values for therapy parameters.

Any discussion of documents, acts, materials, devices, articles or the like which has been included in the present Background is solely for the purpose of providing a context for the present technology. It is not to be taken as an admission that any or all of these matters form part of the prior art base or were common general knowledge in the field relevant to the present technology as it existed before the priority date of each claim of the present disclosure.

### SUMMARY OF THE INVENTION

The present invention seeks to provide a system for programming a neural stimulation device, which will overcome or substantially ameliorate at least some of the deficiencies of the prior art, or at least provide an alternative.

Some implementations herein relate to a system. For example, a neurostimulation system may include a neurostimulation device for controllably delivering neural stimuli, the neurostimulation device having: a stimulus source configured to deliver neural stimuli via selected electrodes of a plurality of implanted electrodes to a neural pathway of a patient; and a control unit configured to control the stimulus source to deliver each neural stimulus according to a stimulus intensity parameter. The neurostimulation system may also include an external computing device in communication with the neurostimulation device, the external computing device having: a display, and a processor configured to: initialise the stimulus intensity parameter; render a stimulation control on the display; render a graphical element adjacent the stimulation control on the display; ramp, on receiving an activation of the stimulation control by a user, a value of the stimulus intensity parameter, while instructing the control unit to control the stimulus source to deliver the neural stimuli according to the ramping value of the stimulus intensity parameter; and cease, upon receiving a de-activation of the stimulation control, ramping the value of the stimulus intensity parameter, wherein the graphical element is configured to: dynamically indicate the ramping value of the stimulus intensity parameter; and indicate a discomfort threshold for the selected electrodes.

Some implementations herein relate to a method. For example, an automated method of controlling a neurostimulation device to deliver neural stimuli using an external computing device in communication with the neurostimulation device may include rendering, by a processor of the external computing device, a stimulation control on a display of the external computing device. The automated method may also include rendering, by the processor, a graphical element adjacent the stimulation control on the display. The automated method may furthermore include ramping, by the processor, on receiving an activation of the stimulation control by a user, a value of a stimulus intensity parameter, while instructing the neurostimulation device to deliver the neural stimuli according to the ramping value of the stimulus intensity parameter via selected electrodes of a plurality of implanted electrodes. The automated method may in addition include ceasing, by the processor, upon receiving a de-activation of the stimulation control, to ramp the value of the stimulus intensity parameter. The automated method may moreover include wherein the graphical element is configured to: dynamically indicate the ramping value of the stimulus intensity parameter; and indicate a discomfort threshold for the selected electrodes. Other embodiments of this aspect include corresponding computer systems, apparatus, and computer programs recorded on one or more computer storage devices, each configured to perform the actions of the automated method.

The present technology has been developed primarily for use in or with neuromodulation of the spinal cord and will be described hereinafter mostly with reference to this application. However, it will be appreciated that the present technology is not limited to this particular field of use, and may be applied in other neuromodulation contexts, including but not limited to sacral nerve stimulation, pudendal nerve stimulation, deep brain stimulation, stimulation of other parts of the peripheral and central nervous system. It will further be appreciated that the present technology may be applied for treatment of conditions other than chronic pain, including but not limited to movement disorders, Crohn's disease, rheumatoid arthritis, diabetes, Reynaud's phenomenon, pelvic floor disorders, chronic inflammatory conditions, migraine, stroke, or depression.

### BRIEF DESCRIPTION OF THE DRAWINGS

Notwithstanding any other implementations which may fall within the scope of the present invention, one or more implementations of the present invention will now be described, by way of example only, with reference to the accompanying drawings, in which:

FIG. 1 schematically illustrates an implanted spinal cord stimulator, according to one implementation of the present technology;

FIG. 2 is a block diagram of the stimulator of FIG. 1;

FIG. 3 is a schematic illustrating interaction of the implanted stimulator of FIG. 1 with a bundle of target nerve fibres;

FIG. 4a illustrates an idealised activation plot for one posture of a patient undergoing neural stimulation;

FIG. 4b illustrates the variation in the activation plots with changing posture of the patient;

FIG. 5 is a schematic illustrating elements and inputs of a closed-loop neural stimulation (CLNS) system, according to one implementation of the present technology;

FIG. 6 illustrates the typical form of an electrically evoked compound action potential (ECAP) of a healthy subject;

FIG. 7 is a block diagram of a neural stimulation therapy system including the implanted stimulator of FIG. 1 according to one implementation of the present technology;

FIG. 8 is an illustration of the stimulus pulses delivered by a stimulation program with four interleaved stimulation sets (stimsets);

FIG. 9a is a schematic illustrating elements of a multi-stimset neural stimulation system implementable by the electronics module of FIG. 2 and suitable for use during programming;

FIG. 9b is a schematic illustrating elements and inputs of a multi-stimset closed-loop neural stimulation (CLNS) system implementable by the electronics module of FIG. 2 and suitable for use during therapy;

FIG. 10a is a schematic diagram illustrating an assisted programming system according to some implementations of the present technology;

FIG. 10b is a flow chart representing an assisted programming workflow implemented by the assisted programming application according to one implementation of the present technology;

FIG. 11 illustrates the locations of the recording and reference electrodes in the six candidate measurement electrode configurations according to one implementation of the present technology;

FIG. 12a illustrates a screen of the user interface display during a patient-controlled stimulus ramp stage of the workflow of FIG. 10b according to one implementation of the present technology;

FIG. 12b illustrates a screen of the user interface display during a patient-controlled stimulus ramp stage of the workflow of FIG. 10b according to one implementation of the present technology;

FIG. 12c illustrates a screen of the user interface display during a patient-controlled stimulus ramp stage of the workflow of FIG. 10b according to one implementation of the present technology;

FIG. 13 is a flowchart illustrating a data collection and analysis method carried out by the APM and the device during the patient-controlled stimulus ramp stage of the workflow of FIG. 10b according to one implementation of the present technology;

FIG. 14a illustrates a screen of the user interface display during a coverage survey stage of the workflow of FIG. 10b according to one implementation of the present technology;

FIG. 14b illustrates a screen of the user interface display during a coverage survey stage of the workflow of FIG. 10b according to one implementation of the present technology;

FIG. 15 is a graph containing a bounded golden growth curve model fitted to a set of value pairs of stimulus intensity and evoked response intensity;

FIG. 16 illustrates a threshold ramp according to one implementation of the present technology;

FIG. 17a illustrates a screen of the user interface display during a coverage selection stage of the workflow of FIG. 10b according to one implementation of the present technology;

FIG. 17b illustrates a screen of the user interface display during a coverage selection stage of the workflow of FIG. 10b according to one implementation of the present technology;

FIG. 17c illustrates a screen of the user interface display during a coverage selection stage of the workflow of FIG. 10b according to one implementation of the present technology;

FIG. 18 is a flowchart illustrating a method of estimating battery single charge life carried out by the APM and the device during the coverage selection stage of the workflow of FIG. 10b according to one implementation of the present technology;

FIG. 19 is a flowchart illustrating a data analysis method carried out by the APM and the device during the coverage selection stage of the workflow of FIG. 10b according to one implementation of the present technology;

FIG. 20 illustrates a posture assessment screen of the user interface display according to one implementation of the present technology; and

FIG. 21 illustrates a program summary screen of the user interface display according to one implementation of the present technology.

### DETAILED DESCRIPTION OF THE PRESENT TECHNOLOGY

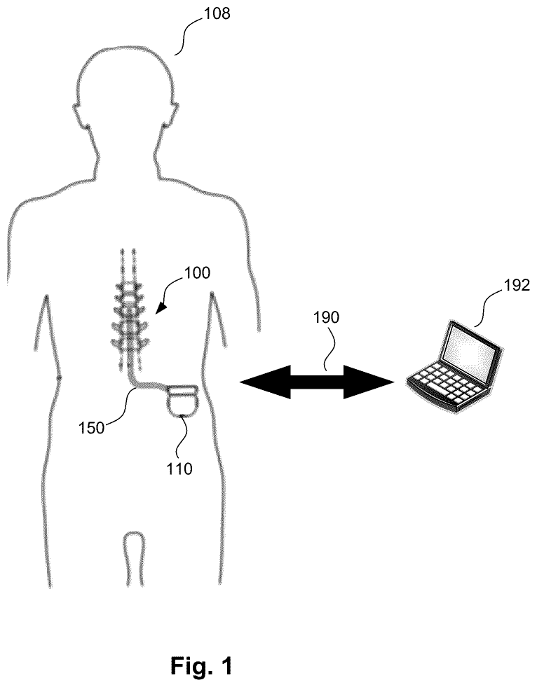

FIG. 1 schematically illustrates an implanted spinal cord stimulator 100 in a patient 108, according to one implementation of the present technology. Stimulator 100 comprises an electronics module 110 housed within a conductive case, implanted at a suitable location. In one implementation, stimulator 100 is implanted in the patient's lower abdominal area or posterior superior gluteal region. In other implementations, the electronics module 110 is implanted in other locations, such as in a flank or sub-clavicularly. The electronics module 110 is configured to electrically connect to an electrode assembly, typically comprising an electrode array 150 implanted within the epidural space and connected to the module 110 by a suitable lead. The electrode array 150 may comprise one or more electrodes such as electrode pads on a paddle lead, circular (e.g., ring) electrodes surrounding the body of a percutaneous lead, conformable electrodes, cuff electrodes, segmented electrodes, or any other type of electrodes capable of forming unipolar, bipolar or multipolar electrode configurations for stimulation and measurement. The electrodes may pierce or affix directly to the tissue itself.

Numerous aspects of the operation of implanted stimulator 100 may be programmable by an external computing device 192, which may be operable by a user such as a clinician or the patient 108. Moreover, implanted stimulator 100 serves a data gathering role, with gathered data being communicated to external device 192 via a transcutaneous communications channel 190. Communications channel 190 may be active on a substantially continuous basis, at periodic intervals, at non-periodic intervals, or upon request from the external device 192. External device 192 may thus provide a clinical interface configured to program the implanted stimulator 100 and recover data stored on the implanted stimulator 100. This configuration is achieved by program instructions collectively referred to as the Clinical Programming Application (CPA) and stored in an instruction memory of the clinical interface.

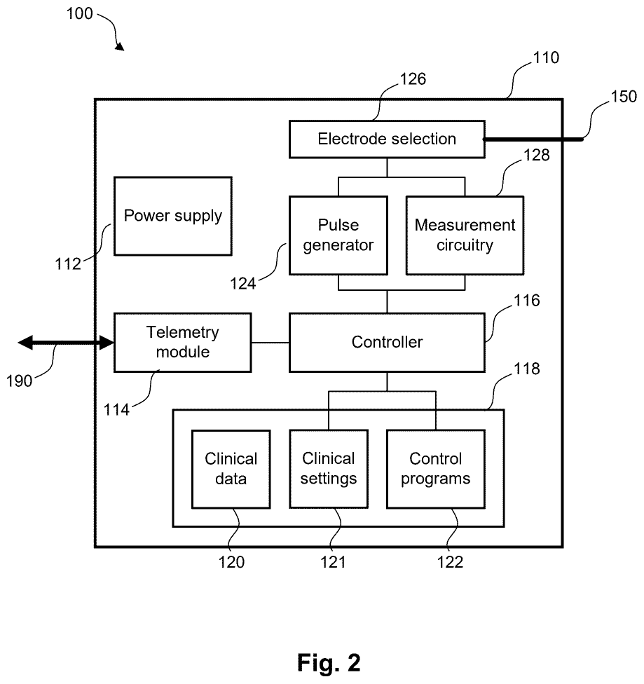

FIG. 2 is a block diagram of the stimulator 100. Electronics module 110 contains a battery 112 and a telemetry module 114. In implementations of the present technology, any suitable type of transcutaneous communications channel 190, such as infrared (IR), radiofrequency (RF), capacitive or inductive transfer, may be used by telemetry module 114 to transfer power or data to and from the electronics module 110 via communications channel 190. Module controller 116 has an associated memory 118 storing one or more of clinical data 120, clinical settings 121, control programs 122, and the like. Controller 116 is configured by control programs 122, sometimes referred to as firmware, to control a pulse generator 124 to generate stimuli, such as in the form of electrical pulses, in accordance with the clinical settings 121. Electrode selection module 126 switches the generated pulses to the selected electrode(s) of electrode array 150, for delivery of the pulses to the tissue surrounding the selected electrode(s). Measurement circuitry 128, which may comprise an amplifier or an analog-to-digital converter (ADC), is configured to process signals comprising neural responses sensed by measurement electrode(s) of the electrode array 150 as selected by electrode selection module 126.

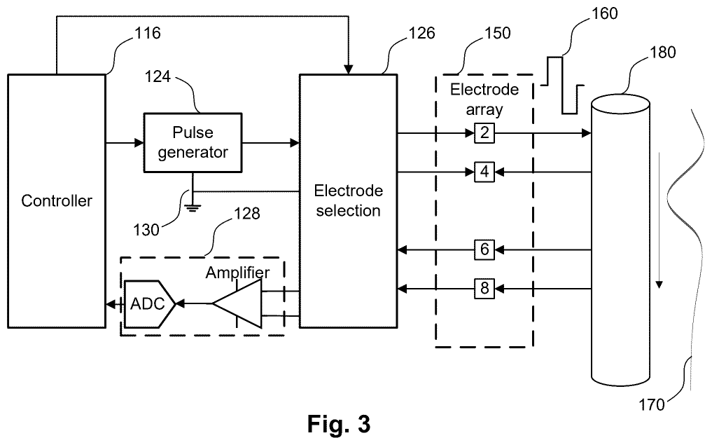

FIG. 3 is a schematic illustrating interaction of the implanted stimulator 100 with a bundle of target nerve fibres 180 in the patient 108. In the implementation illustrated in FIG. 3 the target fibres 180 may be located in the spinal cord, however in alternative implementations the stimulator 100 may be positioned adjacent any target neural tissue including a peripheral nerve, visceral nerve, sacral nerve, parasympathetic nerve, or a brain structure. Electrode selection module 126 selects a stimulus electrode 2 of electrode array 150 through which to deliver a pulse from the pulse generator 124 to surrounding neural tissue including target fibres 180. A pulse may comprise one or more phases, e.g. a monophasic pulse comprises one phase, and a biphasic stimulus pulse 160 comprises two phases. Electrode selection module 126 also selects a return electrode 4 of the electrode array 150 for stimulus current return in each phase, to maintain a zero net charge transfer. An electrode may act as both a stimulus electrode and a return electrode over a complete multiphasic stimulus pulse. The use of two electrodes in this manner for delivering and returning current in each stimulus phase is referred to as bipolar stimulation. Alternative implementations may apply other forms of bipolar stimulation, or may use a greater number of stimulus or return electrodes. By contrast, in monopolar stimulation, current is returned through the conductive case of the stimulator 100, which may therefore be configured and function as an electrode though it is not physically part of the electrode array 150. The set of stimulus electrodes and return electrodes is referred to as the stimulus electrode configuration. Electrode selection module 126 is illustrated as connecting to a ground 130 of the pulse generator 124 to enable stimulus current return via the return electrode 4. However, other connections for current return may be used in other implementations.

Delivery of an appropriate stimulus via electrodes 2 and 4 to the target fibres 180 evokes a neural response 170 comprising an evoked compound action potential (ECAP) which will propagate along the target fibres 180 as illustrated at a rate known as the conduction velocity. The ECAP may be evoked for therapeutic purposes, which in the case of a spinal cord stimulator for chronic pain may be associated with paresthesia at a desired location. To this end, the electrodes 2 and 4 are used to deliver stimuli periodically at any therapeutically suitable stimulus frequency, for example 30 Hz, although other frequencies may be used including frequencies as high as the kHz range. In alternative implementations, stimuli may be delivered in a non-periodic manner such as in bursts, or sporadically, as appropriate for the patient 108. To program the stimulator 100 to the patient 108, a clinician may cause the stimulator 100 to deliver stimuli of various configurations which seek to produce a sensation that may be experienced by the patient as paresthesia. When a stimulus electrode configuration is found which evokes paresthesia in a location and of a size which is congruent with the area of the patient's body affected by pain and of a quality that is comfortable for the patient, the clinician or the patient nominates that configuration for ongoing use. The therapy parameters may be loaded into the memory 118 of the stimulator 100 as the clinical settings 121.

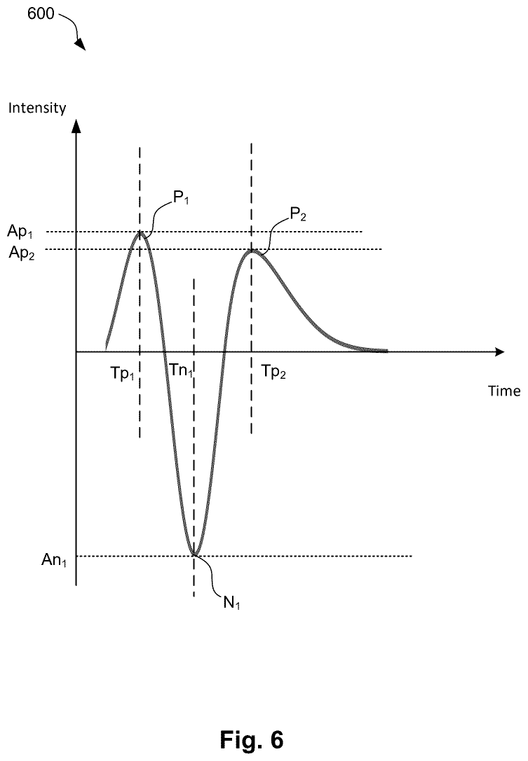

FIG. 6 illustrates the typical form of an ECAP 600 of a healthy subject, as sensed by a single measurement electrode referenced to the system ground 130 or referenced to an indifferent electrode. Such configurations are referred to as single-ended ECAP measurement. The shape and duration of the single-ended ECAP 600 shown in FIG. 6 is predictable because it is a result of the ion currents produced by the ensemble of fibres depolarising and generating action potentials (APs) in response to stimulation. The evoked action potentials (EAPs) generated synchronously among a large number of fibres sum to form the ECAP 600. The ECAP 600 generated from the synchronous depolarisation of a group of similar fibres comprises a positive peak P1, then a negative peak N1, followed by a second positive peak P2. This shape is caused by the region of activation passing the measurement electrode as the action potentials propagate along the individual fibres.

The ECAP may be recorded differentially using two measurement electrodes, as illustrated in FIG. 3. Differential ECAP measurements are less subject to common-mode noise on the surrounding tissue than single-ended ECAP measurements. Depending on the polarity of recording, a differential ECAP may take an inverse form to that shown in FIG. 6, i.e. a form having two negative peaks N1 and N2, and one positive peak P1. Alternatively, depending on the distance between the two measurement electrodes, a differential ECAP may resemble the time derivative of the ECAP 600, or more generally the difference between the ECAP 600 and a time-delayed copy thereof.

The ECAP 600 may be characterised by any suitable characteristic(s) of which some are indicated in FIG. 6. The amplitude of the positive peak P1 is Aₚ₁ and occurs at time Tₚ₁. The amplitude of the positive peak P2 is Aₚ₂ and occurs at time Tₚ₂. The amplitude of the negative peak P1 is Aₙ₁ and occurs at time Tₙ₁. The peak-to-peak amplitude is Aₚ₁+Aₙ₁. A recorded ECAP will typically have a maximum peak-to-peak amplitude in the range of microvolts and a duration of 2 to 3 milliseconds.

The stimulator 100 is further configured to measure the intensity of ECAPs 170 propagating along target fibres 180, whether such ECAPs are evoked by the stimulus from electrodes 2 and 4, or otherwise evoked. To this end, any electrodes of the array 150 may be selected by the electrode selection module 126 to serve as recording electrode 6 and reference electrode 8, whereby the electrode selection module 126 selectively connects the chosen electrodes to the inputs of the measurement circuitry 128. Thus, signals sensed by the measurement electrodes 6 and 8 subsequent to the respective stimuli are passed to the measurement circuitry 128, which may comprise a differential amplifier and an analog-to-digital converter (ADC), as illustrated in FIG. 3. The recording electrode and the reference electrode are referred to as the measurement electrode configuration. The measurement circuitry 128 for example may operate in accordance with the teachings of the above-mentioned International Patent Publication No. WO2012/155183.

Signals sensed by the measurement electrodes 6, 8 and processed by measurement circuitry 128 are further processed by an ECAP detector implemented within controller 116, configured by control programs 122, to obtain information regarding the effect of the applied stimulus upon the target fibres 180. In some implementations, the sensed signals are processed by the ECAP detector in a manner which measures and stores one or more characteristics from each evoked neural response or group of evoked neural responses contained in the sensed signal. In one such implementation, the characteristics comprise a peak-to-peak ECAP amplitude in microvolts (uV). For example, the sensed signals may be processed by the ECAP detector to determine the peak-to-peak ECAP amplitude in accordance with the teachings of International Patent Publication No. WO2015/074121, the contents of which are incorporated herein by reference. Alternative implementations of the ECAP detector may measure and store an alternative characteristic from the neural response, or may measure and store two or more characteristics from the neural response.

Stimulator 100 applies stimuli over a potentially long period such as days, weeks, or months and during this time may store characteristics of neural responses, clinical settings, target response intensity, and other operational parameters in memory 118. To effect suitable sCS therapy, stimulator 100 may deliver tens, hundreds or even thousands of stimuli per second, for many hours each day. Each neural response or group of responses generates one or more characteristics such as a measure of the intensity of the neural response. Stimulator 100 thus may produce such data at a rate of tens or hundreds of Hz, or even kHz, and over the course of hours or days this process results in large amounts of clinical data 120 which may be stored in the memory 118. Memory 118 is however necessarily of limited capacity and care is thus required to select compact data forms for storage into the memory 118, to ensure that the memory 118 is not exhausted before such time that the data is expected to be retrieved wirelessly by external device 192, which may occur only once or twice a day, or less.

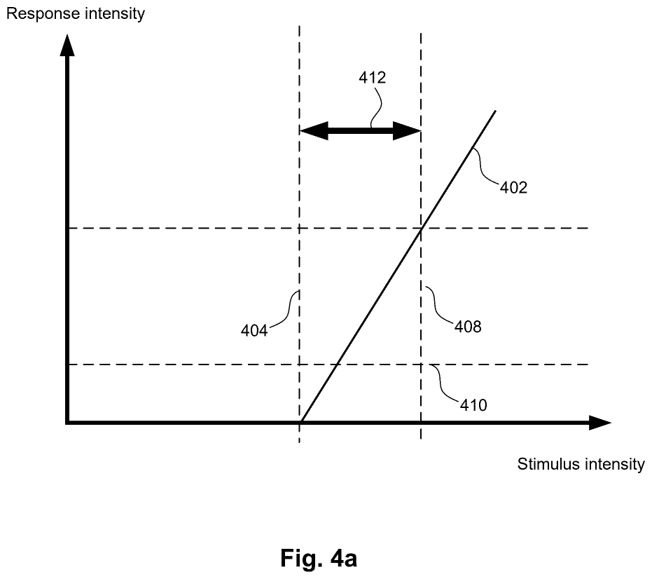

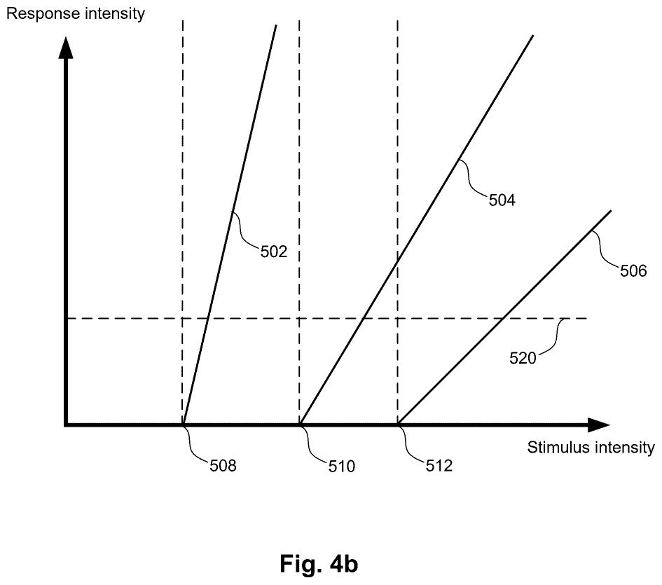

An activation plot, or growth curve, is an approximation to the relationship between stimulus intensity (e.g. an amplitude of the current pulse 160) and intensity of neural response 170 evoked by the stimulus (e.g. an ECAP amplitude). FIG. 4a illustrates an idealised activation plot 402 for one posture of the patient 108. The activation plot 402 shows a linearly increasing ECAP amplitude for stimulus intensity values above a threshold 404 referred to as the ECAP threshold. The ECAP threshold exists because of the binary nature of fibre recruitment; if the field strength is too low, no fibres will be recruited. However, once the field strength exceeds a threshold, fibres begin to be recruited, and their individual evoked action potentials are independent of the strength of the field. The ECAP threshold 404 therefore reflects the field strength at which significant numbers of fibres begin to be recruited, and the increase in response intensity with stimulus intensity above the ECAP threshold reflects increasing numbers of fibres being recruited. Below the ECAP threshold 404, the ECAP amplitude may be taken to be zero. Above the ECAP threshold 404, the activation plot 402 has a positive, approximately constant slope indicating a linear relationship between stimulus intensity and the ECAP amplitude. Such a relationship may be modelled in piecewise linear form as:

$$d = \{ \begin{matrix}S ( s - T ) , & s \geq T \\ 0 , & s < T\end{matrix} \qquad ( 1 )$$

where s is the stimulus intensity, d is the ECAP amplitude, T is the ECAP threshold and S is the slope of the activation plot (referred to herein as the patient sensitivity) above the ECAP threshold T. The sensitivity S and the ECAP threshold T are the key parameters of the activation plot 402.

FIG. 4a also illustrates a discomfort threshold 408, which is a stimulus intensity above which the patient 108 experiences uncomfortable or painful stimulation. FIG. 4a also illustrates a perception threshold 410. The perception threshold 410 corresponds to an ECAP amplitude that is barely perceptible by the patient. There are a number of factors which can influence the position of the perception threshold 410, including the posture of the patient. Perception threshold 410 may correspond to a stimulus intensity that is greater than the ECAP threshold 404, as illustrated in FIG. 4a, if patient 108 does not perceive low levels of neural activation. Conversely, the perception threshold 410 may correspond to a stimulus intensity that is less than the ECAP threshold 404, if the patient has a high perception sensitivity to lower levels of neural activation than can be detected in an ECAP, or if the signal-to-noise ratio of the ECAP is low.

For effective and comfortable operation of an implantable neuromodulation device such as the stimulator 100, it is desirable to maintain stimulus intensity within a therapeutic range. A stimulus intensity within a therapeutic range 412 is above the ECAP threshold 404 and below the discomfort threshold 408. In principle, it would be straightforward to measure these limits and ensure that stimulus intensity, which may be closely controlled, always falls within the therapeutic range 412. However, the activation plot, and therefore the therapeutic range 412, varies with the posture of the patient 108.

FIG. 4b illustrates the variation in the activation plots with changing posture of the patient. A change in posture of the patient may cause a change in impedance of the electrode-tissue interface or a change in the distance between electrodes and the spinal cord. While the activation plots for only three postures, 502, 504 and 506, are shown in FIG. 4b, the activation plot for any given posture can lie between or outside the activation plots shown, on a continuously varying basis depending on posture. Consequently, as the patient's posture changes, the ECAP threshold changes, as indicated by the ECAP thresholds 508, 510, and 512 for the respective activation plots 502, 504, and 506. Additionally, as the patient's posture changes, the patient sensitivity also changes, as indicated by the varying slopes of activation plots 502, 504, and 506. In general, as the distance between the stimulus electrodes and the spinal cord increases, the ECAP threshold increases and the sensitivity decreases. The activation plots 502, 504, and 506 therefore correspond to increasing distance between stimulus electrodes and spinal cord, and decreasing patient sensitivity.

To keep the applied stimulus intensity within the therapeutic range as patient posture varies, in some implementations an implantable neuromodulation device such as the stimulator 100 may adjust the applied stimulus intensity based on a feedback variable that is determined from one or more measured ECAP characteristics. In one implementation, the device may adjust the stimulus intensity to maintain the measured ECAP amplitude at or near a target response intensity. For example, the device may calculate an error between a target ECAP amplitude and a measured ECAP amplitude, and adjust the applied stimulus intensity to bring the measured ECAP amplitude closer to the target ECAP amplitude, such as by adding the scaled error to the current stimulus intensity. A neuromodulation device that operates by adjusting the applied stimulus intensity to maintain a feedback variable at or near a target value is said to be operating in closed-loop mode and will also be referred to as a closed-loop neural stimulation (CLNS) device. By adjusting the applied stimulus intensity to maintain the measured ECAP amplitude at or near an appropriate target response intensity, such as a target ECAP amplitude 520 illustrated in FIG. 4b, a CLNS device will generally keep the stimulus intensity within the therapeutic range as patient posture varics.

A CLNS device comprises a pulse generator that takes a stimulus intensity value and converts it into neural stimuli comprising a sequence of electrical pulses according to a predefined stimulation pattern. The stimulation pattern is parametrised by multiple stimulus parameters including stimulus amplitude, pulse width, number of phases, order of phases, number of stimulus electrode poles (two for bipolar, three for tripolar etc.), and stimulus rate or frequency. At least one of the stimulus parameters, for example the stimulus amplitude, is controlled by the pulse generator to implement the received stimulus intensity value. For example, all stimulus parameters may be held constant except stimulus amplitude which is determined in proportion to the received stimulus intensity value. Alternatively, all stimulus parameters may be held constant except pulse width which is varied in proportion to the desired stimulus intensity.

In an example CLNS system, the user sets a target response intensity, and the CLNS device performs proportional-integral-differential (PID) control. In some implementations, the differential and proportional contributions are disregarded and the CLNS device uses a first order integrating feedback loop. The pulse generator generates a stimulus in accordance with a stimulus intensity parameter, which evokes a neural response in the patient. The intensity of an evoked neural response (e.g. an ECAP) is measured by the CLNS device and compared to the target response intensity.

The measured neural response intensity, and its deviation from the target response intensity, is used by the feedback loop to determine possible adjustments to the stimulus intensity parameter to maintain the neural response at or near the target response intensity. If the target response intensity is properly chosen, the patient receives consistently comfortable and therapeutic stimulation through posture changes and other perturbations to the stimulus/response behaviour.

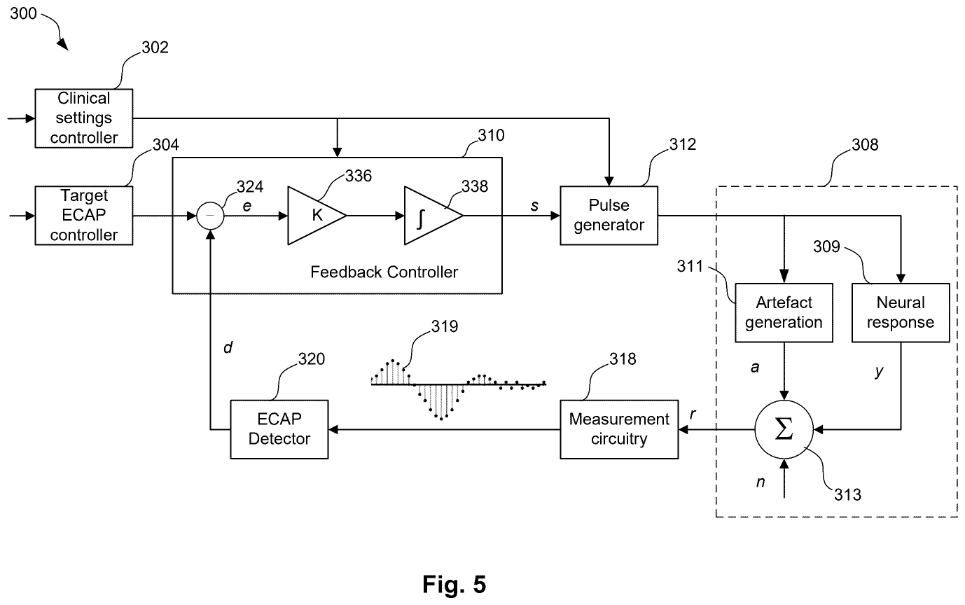

FIG. 5 is a schematic illustrating elements and inputs of a closed-loop neural stimulation (CLNS) system 300, according to one implementation of the present technology. The system 300 comprises a pulse generator 312 which converts a stimulus intensity parameter s, in concert with a set of predefined stimulus parameters, into neural stimuli comprising a sequence of electrical pulses delivered via the stimulus electrodes (not shown in FIG. 5). According to one implementation, the predefined stimulus parameters comprise the number and order of phases, the number of stimulus electrode poles, the pulse width, and the stimulus rate or frequency, and the pulse generator 312 determines the stimulus amplitude in proportion to the stimulus intensity parameter s.

The generated stimulus crosses from the electrodes to the spinal cord, which is represented in FIG. 5 by the dashed box 308. The box 309 represents the evocation of a neural response y by the stimulus as described above. The box 311 represents the evocation of an artefact signal a, which is dependent on stimulus intensity and other stimulus parameters, as well as the electrical environment of the measurement electrodes. Various sources of measurement noise n, as well as the artefact a, may add to the evoked response y at the summing element 313 to form the sensed signal r, including: electrical noise from external sources such as 50 Hz mains power; electrical disturbances produced by the body such as neural responses evoked not by the device but by other causes such as peripheral sensory input; EEG; EMG; and electrical noise from measurement circuitry 318.

The neural recruitment arising from the stimulus is affected by mechanical changes, including posture changes, walking, breathing, heartbeat and so on. Mechanical changes may cause impedance changes, or changes in the location and orientation of the nerve fibres relative to the electrode array(s). As described above, the intensity of the evoked response provides a measure of the recruitment of the fibres being stimulated. In general, the more intense the stimulus, the more recruitment and the more intense the evoked response. An evoked response typically has a maximum amplitude in the range of microvolts, whereas the voltage resulting from the stimulus applied to evoke the response is typically several volts.

Measurement circuitry 318, which may be identified with measurement circuitry 128, amplifies the sensed signal r (potentially including evoked neural response, artefact, and measurement noise), and samples the amplified sensed signal r to capture a “signal window” 319 comprising a predetermined number of samples of the amplified sensed signal r. The ECAP detector 320 processes the signal window 319 and outputs a measured neural response intensity d. In one implementation, the neural response intensity comprises a peak-to-peak ECAP amplitude. The measured response intensity d (an example of a feedback variable) is input into the feedback controller 310. The feedback controller 310 comprises a comparator 324 that compares the measured response intensity d to a target ECAP amplitude as set by the target ECAP controller 304 and provides an indication of the difference between the measured response intensity d and the target ECAP amplitude. This difference is the error value, e.

The feedback controller 310 calculates an adjusted stimulus intensity parameter, s, with the aim of maintaining a measured response intensity d equal to the target ECAP amplitude. Accordingly, the feedback controller 310 adjusts the stimulus intensity parameter s to minimise the error value, e. In one implementation, the controller 310 utilises a first order integrating function, using a gain element 336 and an integrator 338, in order to provide suitable adjustment to the stimulus intensity parameter s. According to such an implementation, the current stimulus intensity parameter s may be determined by the feedback controller 310 as

$$s = \int K e d t \qquad ( 2 )$$

where K is the gain of the gain element 336 (the controller gain). This relation may also be represented as

$$δ s = K e \qquad ( 3 )$$

where δs is an adjustment to the current stimulus intensity parameter s.

A target ECAP amplitude is input to the feedback controller 310 via the target ECAP controller 304. In one implementation, the target ECAP controller 304 provides an indication of a specific target ECAP amplitude. In another implementation, the target ECAP controller 304 provides an indication to increase or to decrease the present target ECAP amplitude. The target ECAP controller 304 may comprise an input into the CLNS system 300, via which the patient or clinician can input a target ECAP amplitude, or indication thereof. The target ECAP controller 304 may comprise memory in which the target ECAP amplitude is stored, and from which the target ECAP amplitude is provided to the feedback controller 310.

A clinical settings controller 302 provides clinical settings to the system 300, including the feedback controller 310 and the pulse generator 312. In one example, the clinical settings controller 302 may be configured to adjust the controller gain K of the feedback controller 310 to adapt the feedback loop to patient sensitivity. The clinical settings controller 302 may comprise an input into the CLNS system 300, via which the patient or clinician can adjust the clinical settings. The clinical settings controller 302 may comprise memory in which the clinical settings are stored, and are provided to components of the system 300.

In some implementations, two clocks (not shown) are used, being a stimulus clock operating at the stimulus frequency (e.g. 60 Hz) and a sample clock for sampling the sensed signal r (for example, operating at a sampling frequency of 16 kHz). As the ECAP detector 320 is linear, only the stimulus clock affects the dynamics of the CLNS system 300. On the next stimulus clock cycle, the pulse generator 312 generates a stimulus in accordance with the adjusted stimulus intensity s. Accordingly, there is a delay of one stimulus clock cycle before the stimulus intensity is updated in light of the error value e.

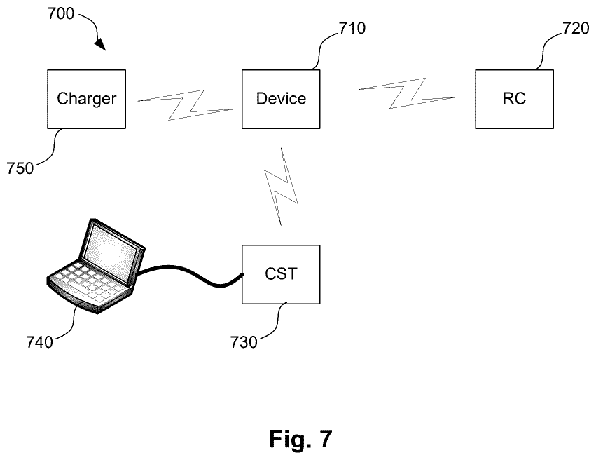

FIG. 7 is a block diagram of a neural stimulation system 700. The neural stimulation system 700 is centred on a neuromodulation device 710. In one example, the neuromodulation device 710 may be implemented as the stimulator 100 of FIG. 1, implanted within a patient (not shown). The neuromodulation device 710 is connected wirelessly to a remote controller (RC) 720. The remote controller 720 is a portable computing device that provides the patient with control of their stimulation in the home environment by allowing control of the functionality of the neuromodulation device 710, including one or more of the following functions: enabling or disabling stimulation; adjustment of stimulus intensity or target response intensity; and selection of a stimulation control program from the control programs stored on the neuromodulation device 710.

The charger 750 is configured to recharge a rechargeable power source of the neuromodulation device 710. The recharging is illustrated as wireless in FIG. 7 but may be wired in alternative implementations.

The neuromodulation device 710 is wirelessly connected to a Clinical System Transceiver (CST) 730. The wireless connection may be implemented as the transcutaneous communications channel 190 of FIG. 1. The CST 730 acts as an intermediary between the neuromodulation device 710 and the Clinical Interface (CI) 740, to which the CST 730 is connected. A wired connection is shown in FIG. 7, but in other implementations, the connection between the CST 730 and the CI 740 is wireless.

The CI 740 may be implemented as the external computing device 192 of FIG. 1. The CI 740 is configured to program the neuromodulation device 710 and recover data stored on the neuromodulation device 710. This configuration is achieved by program instructions collectively referred to as the Clinical Programming Application (CPA) and stored in an instruction memory of the CI 740.

### Multi-Stimset Neural Stimulation

For some patients, it is beneficial for a neural stimulation therapy program to comprise multiple stimulation sets. A stimulation set (“stimset”) is a set of stimulus and return electrodes, or more precisely a stimulus electrode configuration (SEC), along with the stimulus parameters that govern the stimulation pulses delivered via that SEC.

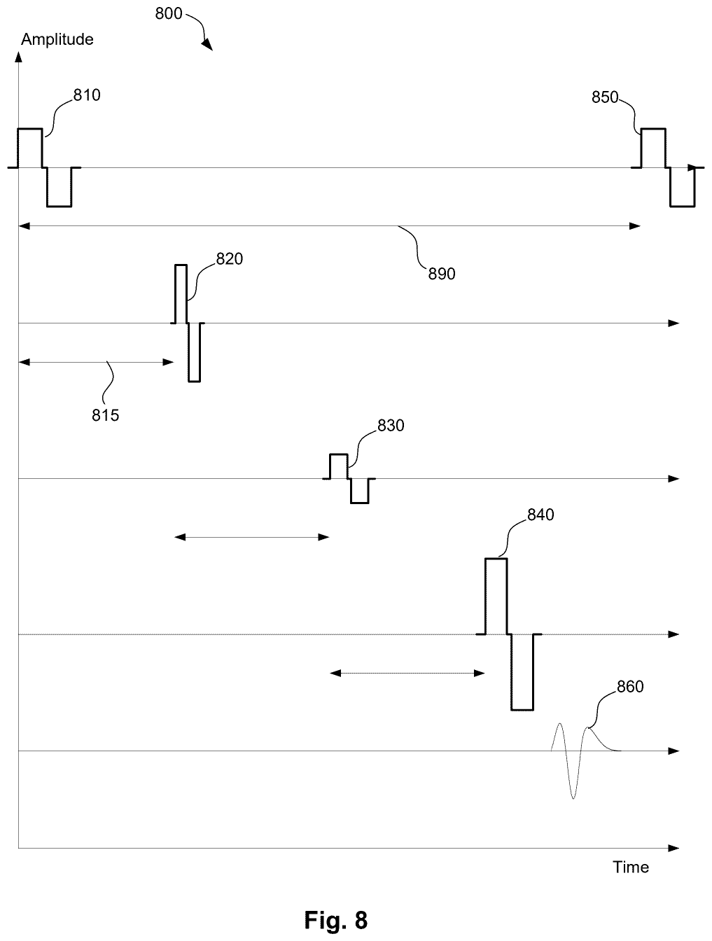

FIG. 8 is an illustration 800 of the stimulus pulses delivered by a stimulation program with four interleaved stimsets. The stimulus pulse train delivered according to each stimset is illustrated on a separate, but vertically aligned, horizontal axis representing time. All the stimulus pulse trains are delivered at the same stimulus frequency. (It is not a requirement that all the stimulus pulse trains for the respective stimsets are delivered at the same stimulus frequency; however it is so represented in FIG. 8 for case of illustration.) The first stimulus pulse 810, delivered according to the first stimset, is illustrated as a biphasic, anodic-first stimulus pulse, though many other stimulus pulse types are contemplated. The second, third, and fourth stimulus pulses 820, 830, and 840, delivered according to the second, third, and fourth stimsets in the program respectively, are also biphasic, anodic-first stimulus pulses with different pulse widths and different amplitudes. Each stimulus pulse is illustrated as delayed in time by a constant amount (the inter-stimulus interval, or ISI, 815) from the stimulus pulse delivered according to the preceding stimset. However, this is not to be interpreted as limiting, since the intervals between the pulses in the various stimsets may be different. Because all the stimulus pulse trains in FIG. 8 are delivered at the same stimulus frequency, the four stimulus pulses 810, 820, 830, 840 form a cycle that repeats indefinitely without any change to the relative timing of the pulses from the different stimsets. The fifth stimulus pulse 850 is a subsequent pulse in the pulse train delivered according to the first stimset and is therefore illustrated on the same time axis as the first stimulus pulse 810, and the cycle repeats thereafter. The stimulus period 890 is the period of repetition of the full cycle and is equal to the reciprocal of the stimulus frequency. In one implementation, the ISI 815 is the stimulus period divided by the number of stimsets, so that the stimuli are evenly spaced throughout the stimulus period 890.

Also illustrated is an evoked neural response in the form of an evoked compound action potential (ECAP) 860 as sensed by a predetermined measurement electrode configuration (MEC) on a common time axis with the stimulus pulses. The illustrated ECAP 860 is evoked by the fourth stimulus pulse 840. A closed-loop neural stimulation (CLNS) system programmed with multiple interleaved stimsets, as illustrated in FIG. 8, may be based on measurements of the ECAP 860. That is to say, closed-loop adjustments to the stimulus parameters of all stimsets may all be based on measurements of the ECAP 860 evoked by a single stimset, referred to as the primary stimset. In FIG. 8, the final stimset in the cycle is the primary stimset.

If the ISI 815 is greater than the refractory period and sufficiently long that ECAPs evoked by the earlier stimsets are not obscured by stimulus crosstalk and artefact from the other stimulus pulses in the cycle, any or all of the stimsets in the cycle may evoke a measurable ECAP. In such implementations, an ECAP may be measured from each stimset in the cycle.

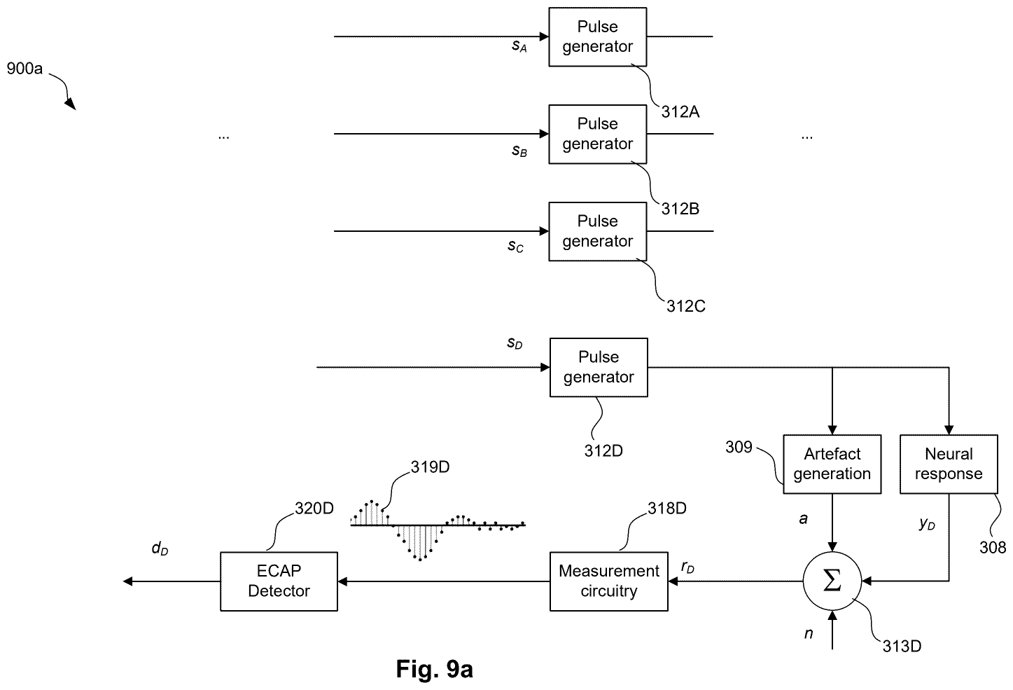

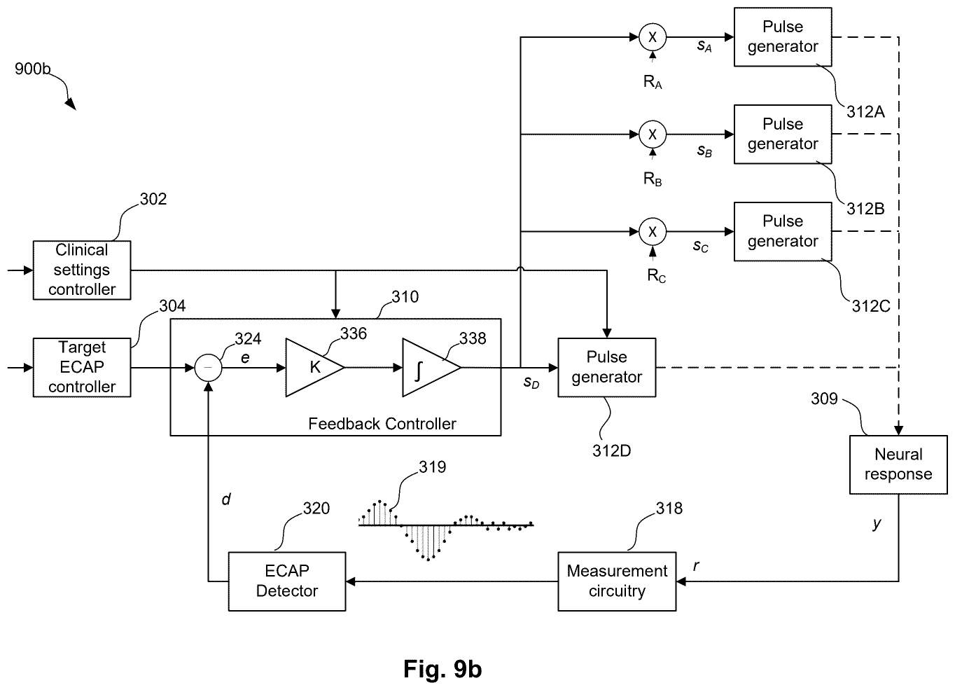

FIG. 9a is a schematic illustrating elements of a multi-stimset neural stimulation system 900a implementable by the electronics module 110. The multi-stimset neural stimulation system 900a is the same as the neural stimulation system 300 of FIG. 5, with like numbers indicating like elements, without the feedback controller 310 and with the addition of three further stimsets. The four stimsets are labelled A, B, C, and D and are delivered by pulse generators 312A, 312B, 312C, and 312D (each of which corresponds to the pulse generator 312 in the CLNS system 300) according to respective stimulus intensity parameters s_{A}, s_{B}, s_{C}, and s_{D}, and via respective SECs. The pulses delivered by the pulse generators 312A, 312B, 312C, and 312D correspond to the stimulus pulses 810, 820, 830, and 840 of FIG. 8. FIG. 9a illustrates that a neural response yo is evoked from stimset D and combined with artefact a and noise n at the summing element 313D to become the sensed signal r_{D} at an MEC corresponding to stimset D. Measurement circuitry 318D amplifies and samples the sensed signal r to capture a signal window 319D. The ECAP detector 320D processes the signal window 319D and outputs a measured neural response intensity d_{D}. The measurement circuitry 318D and ECAP detector 320D are replicated for the other three stimsets (not illustrated) to yield signal windows 319A, 319B, and 319C and measured neural response intensities da, de, and de from respective MECs for stimsets A, B, and C respectively. The measured neural response intensities da, de, dc, and do may be used by one or more feedback controllers (not illustrated) within the system 900a for the stimsets or transmitted to the APM 1040 for analysis. Alternatively, as described above, the signal windows 319A, 319B, 319C, and 319D may be stored temporarily in memory 118 before being analysed or transmitted to the APM 1040 for analysis, bypassing the ECAP detectors.

The implementation 900a of a multi-stimset neural stimulation system is suitable for use to gather data during programming.

FIG. 9b is a schematic illustrating elements and inputs of a multi-stimset closed-loop neural stimulation (CLNS) system 900b implementable by the electronics module 110 and suitable for usc during therapy. The multi-stimset CLNS system 900b is the same as the neural stimulation system 300 of FIG. 5, with like numbers indicating like elements, with the addition of three further stimsets. The noise and artefact in FIG. 5 have been omitted from FIG. 9b for clarity. The four stimsets are labelled A, B, C, and D and are delivered by stimulators 312A, 312B, 312C, and 312D according to respective stimulus intensity parameters s_{A}, s_{B}, s_{C}, and sp, and via respective SECs. The neural response y may be evoked by any of stimsets A, B, C, and D, which is why the neural response box 309 is joined by dashed lines to all four stimulators 312A, 312B, 312C, and 312D in FIG. 9b. The measured neural response intensity d of the evoked response y is used by the feedback controller 310 to control all four stimulus intensity parameters s_{A}, s_{B}, s_{C}, and s_{D}.

In the implementation of FIG. 9b, the stimulus intensity parameter s_{D} for stimset D is the largest of the four stimulus intensity parameters s_{A}, s_{B}, s_{C} and s_{D} and is the stimulus intensity parameter that is directly adjusted by the feedback controller 310. The stimulus intensity parameter s_{D} is scaled by ratios R_{A}, R_{B}, and R_{C} to obtain the stimulus intensity parameters s_{A}, s_{B}, and s_{C} for stimsets A, B, and C respectively at the end of each cycle. The ratios R_{A}, R_{B}, and R_{C}, which are all less than or equal to one, are fixed at the ratios of the respective stimulus intensities s_{A}, s_{B}, and s_{C} at which the respective stimsets were originally programmed, to the originally programmed stimulus intensity s_{D} of the largest stimset D and form part of the clinical settings 121 of the multi-stimset program. In such an implementation, the stimulus intensity parameters s_{A}, s_{B}, and s_{C} always remain in fixed ratio with the stimulus intensity parameter s_{D} and with each other. This is referred to as ratiometric adjustment. So for example, if the originally programmed stimulus intensities were 1 mA, 2 mA, 4 mA, and 6 mA for the four stimsets A, B, C, and D respectively, the ratios R_{A}, R_{B}, and Re are fixed at programming time at 1/6, 1/3, and 2/3 respectively. If during therapy the feedback controller 310 adjusts the largest stimset intensity parameter s_{D} to 6.6 mA, the stimulus intensity parameters s_{A}, s_{B}, and s_{C} are automatically adjusted to 1.1 mA, 2.2 mA, and 4.4 mA respectively. The clinical settings controller 302 provides the respective stimulus parameters to the pulse generators 312A, 312B, 312C, and 312D.

It may be seen from FIG. 9b that the adjustments to the stimulus intensity parameters after each stimulus cycle are all in fixed proportion. A ratiometric multi-stimset CLNS system therefore emulates a CLNS system with four separate feedback loops driven by the four stimsets, wherein each loop has the same controller gain. A ratiometric multi-stimset CLNS system is effective to maintain the responses evoked by each stimset at a constant neural response intensity on the condition that when the patient moves to a new posture, the threshold and slope of all activation plots move in a proportional manner.

### A Measure of Neural Activation

Neural activation refers to the number of fibres recruited by a stimulus provided by a neural stimulator. The stimulus current flowing through the one or more stimulus electrodes to the one or more return electrodes generates a field at the neural tissue (e.g. the spinal cord). It will be appreciated that for a given stimulus intensity, as the stimulus electrode changes distance with respect to the cord, the field intensity at the neural tissue changes. The stimulus intensity at which the field generated by the stimulus begins to recruit fibres, for a specific posture of a patient, is the activation threshold (or ECAP threshold) for that posture of the patient.

The recruitment of fibres begins at approximately the same field intensity at the spinal cord regardless of the patient's posture. Therefore, the ECAP threshold for a patient's current posture provides a calibration point for estimating the neural activation resulting from stimuli at a given stimulus intensity. At stimulus intensities above the ECAP threshold, the neural activation of the field generated by the stimulus increases with the ratio by which the stimulus intensity exceeds the ECAP threshold. A measure D of neural activation resulting from stimuli at a stimulus intensity s may therefore be determined as the “dose ratio” of stimulus intensity s to the ECAP threshold s_{T}.

$$D = \frac{s}{{s}_{T}} \qquad ( 4 )$$

It has been hypothesised that the dose ratio defined by equation (4) is a measure of dose that is normalised across all patients, i.e. delivery of a similar dose will have a similar analgesic effect on different patients, and on the same patient in different postures. It has further been demonstrated that dose ratio D is correlated with patient analgesic effect and that a dose ratio of approximately 1.4 is a threshold of therapeutic efficacy across many patients.

A more general measure of neural activation may be determined as follows:

$$λ = 1 + \frac{s - {s}_{T}}{{s}_{T}^{γ}} \qquad ( 5 )$$

where the exponent y is a patient characteristic. The measure A defined by equation (5) is referred to herein as a dose metric because in the special case in which y equals one, equation (5) degenerates to equation (4) defining dose ratio D.

### Assisted Programming System

As mentioned above, obtaining patient feedback about their sensations is important during programming of closed-loop neural stimulation therapy, but mediation by trained clinical engineers is expensive and time-consuming. It would therefore be advantageous if patients could program their own implantable device themselves, or with some assistance from a clinician. However, interfaces for current programming systems are non-intuitive and generally unsuitable for direct use by patients because of their technical nature. The Assisted Programming System (APS) described herein is a programming system that is as intuitive for non-technical users as possible while avoiding discomfort to the patient.

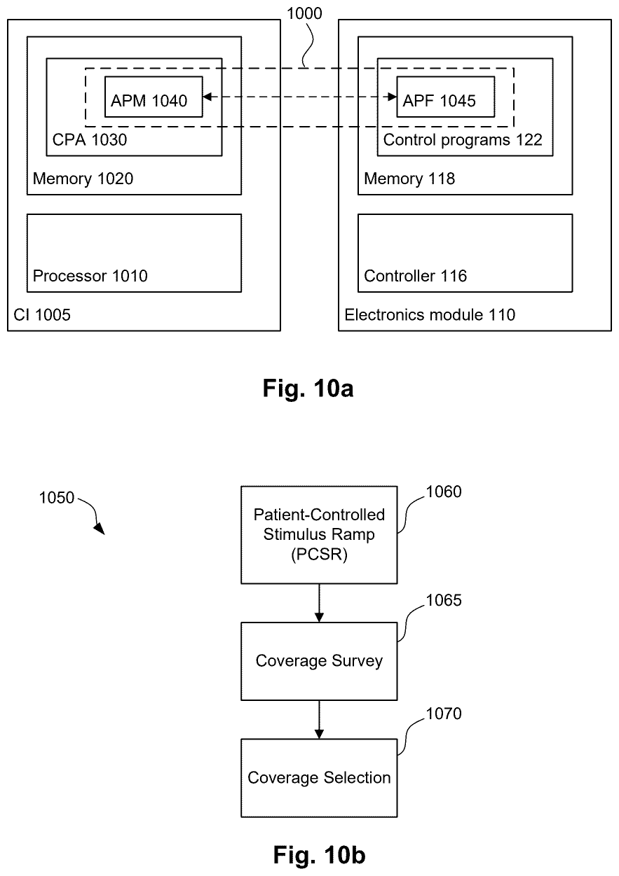

FIG. 10a is a schematic diagram illustrating an APS 1000 according to some implementations of the present technology. The APS 1000 comprises two elements in communication with each other: an Assisted Programming Module (APM) 1040 and an Assisted Programming Firmware (APF) 1045. The APF 1045 forms part of the control programs (firmware) 122 stored in the memory 118 of the electronics module 110 of the stimulator 100 (not shown) in order to configure the controller 116 of the electronics module 110. The APM 1040 forms part of the CPA 1030 which is stored in the instruction memory 1020 of the clinical interface 1005 in order to configure the processor 1010 of the clinical interface 1005. The clinical interface 1005, which is an implementation of the CI 740 of FIG. 7, is in communication with the electronics module 110 possibly via a CST (not shown in FIG. 10a) such as the CST 730 of FIG. 7. The APF 1045 is configured to complement the operation of the APM 1040 by responding to commands issued by the APM 1040 to the electronics module 110 to deliver specified stimuli to the target neural tissue, and by returning data comprising measurements of neural response characteristics to the delivered stimuli. The data obtained from the electronics module 110 under the control of the APF 1045 is analysed by the APM 1040 to determine the clinical settings 121 for the neural stimulation therapy to be delivered by the electronics module 110.

In other implementations of the present technology, all the processing of the APS 1000 is done by the APF 1045. In other words, the data obtained from the patient is not passed to the APM 1040, but is analysed by the controller 116 of the electronics module 110, configured by the APF 1045, to determine the clinical settings 121 for the neural stimulation therapy to be delivered by the electronics module 110.

In some implementations of the APS 1000 in which the APM 1040 analyses the data from the patient, the APS 1000 instructs the electronics module 110 to capture and return signal windows to the CI 1000. In such implementations, the electronics module 110 captures the signal windows using the measurement circuitry 128 and bypasses the ECAP detector 320, storing the data representing the raw signal windows temporarily in memory 118 before transmitting the data representing the captured signal windows to the APM 1040 for analysis.

Following the programming, the APS 1000 may load the determined program onto the electronics module 110 to govern subsequent neural stimulation therapy. In one implementation, the program comprises clinical settings 121, also referred to as therapy parameters, that are input to the electronics module 110 and stored in the clinical settings controller 302. The patient may subsequently control the electronics module 110 to deliver and adjust the therapy according to the determined program using a remote controller for the electronics module 110 such as the remote controller 720 as described above. The determined program may also, or alternatively, be loaded into the CPA 1030 for validation and modification. Validation and modification of the determined program may also be carried out by the APS 1000 itself.

FIG. 10b is a flow chart representing an assisted programming workflow 1050 implemented by the APM, according to one implementation of the present technology. In the assisted programming workflow 1050, control of the CI 740 is handed over to a user, for example the patient, who interacts with the APM for the entirety of the workflow. In some implementations, the patient remains in a fixed predetermined posture throughout the workflow. Having direct patient involvement allows for faster feedback because subjective responses to stimulation do not have to be communicated via a clinician. However, the workflow 1050 is just one possible implementation of an APM, and it should be noted that there is no formal requirement for any part of the assisted programming system to include direct patient involvement.

The workflow 1050 has several stages: a Patient-Controlled Stimulus Ramp (PCSR) stage 1060, an (optional) Coverage Survey stage 1065, and a Coverage Selection stage 1070.

The PCSR stage 1060 is configured to deliver stimuli of a gradually increasing intensity via each of one or more candidate stimulus electrode configurations (SECs) and receive subjective input from the patient as to a maximum value of stimulus intensity (“Max” value) for that SEC. The Max value may be identified with the discomfort threshold 408 of FIG. 4a. Meanwhile, the APM is configured to record sensed signals at each of multiple measurement electrode configurations (MECs) for each candidate SEC. The PCSR stage 1060 is configured to then choose a suitable MEC for each candidate SEC based on the sensed signal data. The PCSR stage 1060 is described in more detail below.

The Coverage Survey stage 1065 is configured to determine a comfortable stimulus intensity for each candidate SEC. The comfortable stimulus intensity is determined for each candidate SEC based on the Max and ECAP Threshold value for the SEC. The coverage survey stage 1065 is then configured to receive input from the patient concerning their sensations in response to stimuli delivered via each candidate SEC at the corresponding comfortable stimulus intensity. Based on the patient input, the comfortable stimulus intensity at each candidate SEC may be adjusted. In addition, if stimulus delivered via any candidate SEC feels uncomfortable to the patient in an area of the body, the candidate SEC itself may be adjusted and the PCSR stage 1060 may be repeated for the adjusted SEC. The Coverage Survey stage 1065 is described in more detail below.

The PCSR stage 1060 and the Coverage Survey stage 1065 is repeated for each candidate SEC. After all candidate SECs have been completed, resulting in a set of successful candidate SECs, each with a comfortable stimulus intensity and a corresponding MEC, the workflow 1050 proceeds to the Coverage Selection stage 1070.

The Coverage Selection stage 1070 is configured to receive input from the patient to select one or more of the successful candidate SECs. The comfortable stimulus intensity delivered via each candidate SEC during the Coverage Selection stage 1070 is based on the comfortable stimulus intensity derived for that SEC at the Coverage Survey stage 1065. The coverage selection stage 1070 allows the patient to test different combinations of candidate SECs before selecting which ones to keep for their final program.

The Coverage Selection stage 1070 is then configured to choose a primary SEC/MEC combination from among the selected candidate SECs based on the data gathered during the PCSR stage 1060. If such a choice is successfully made, the Coverage Selection stage 1070 is configured to calculate therapy parameters for the primary SEC/MEC combination. The selected SECs, including the primary SEC/MEC combination, and the calculated therapy parameters are referred to as the determined program. The Coverage Selection stage 1070 is described in more detail below.

In the workflow 1050, the APM may use predetermined values of certain therapy parameters. In one implementation, those parameters and values are:
Stimulus frequency: 40 Hz.
Pulse width: 240 microseconds
Inter-phase gap: 200 microseconds
Pulse shape: triphasic, with anodic phase first
Signal window length: 60 samples
Sampling frequency: 16 kHz
Inter-stimulus interval: 5 milliseconds

In one implementation of the workflow 1050, four default candidate stimulus electrode configurations (SECs) (labelled “A”, “B”, “C”, and “D” for UI purposes) are defined. Each SEC is tripolar, comprising a stimulus electrode that acts primarily as a cathode, sinking stimulus current, with the two neighbouring return electrodes on either side of the stimulus electrode acting primarily as anodes, sourcing return currents. Tripolar stimulus electrode configurations are described in more detail in International Patent Publication no. WO2017/219096 by the present applicant, the entire contents of which are herein incorporated by reference.

In some implementations, the APM defines the default candidate SECs on the assumption that the electrode array 150 consists of two percutaneous leads implanted approximately symmetrically to left and right (as viewed from behind the patient) of the patient's midline, as illustrated in FIG. 1 for one lead. In one implementation, each lead comprises twelve contacts (electrodes), numbered such that a contact index of zero is the topmost (rostral) contact of a lead and contact index 11 is the bottom-most (caudal) contact of a lead. The stimulus electrodes in each of the four candidate SECs are defined as follows: top left (contact index 1, left lead) (labelled as SEC “A”), top right (contact index 1, right lead) (labelled as SEC “B”), bottom left (contact index 10, left lead) (labelled as SEC “C”), and bottom right (contact index 10, right lead) (labelled as SEC “D”). In other implementations with a different number of contacts in each lead, the bottom left and bottom right stimulus electrodes are defined to be the second-most caudal contact on the respective leads.

The APM may define other default candidate SECs suitable for other configurations of the electrode array 150. For example, if the electrode array 150 is configured as a paddle lead, the four candidate SECs may be defined as the four electrode tripoles located at the top left, top right, bottom left, and bottom right on the paddle lead.

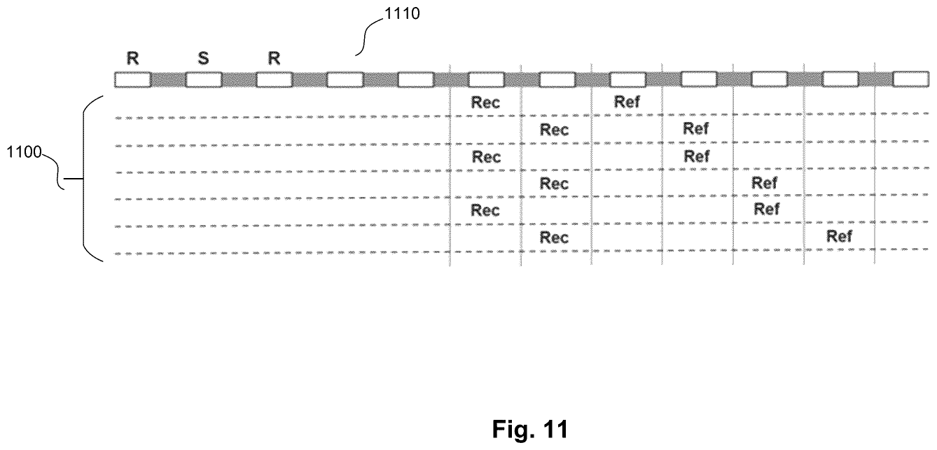

For each SEC, the APM defines multiple candidate measurement electrode configurations (MECs). An MEC comprises two electrodes for differential ECAP recording, as illustrated in FIG. 3. The measurement electrode connected to the positive terminal of the measurement circuitry 318 is referred to as the recording electrode, while the measurement electrode connected to the negative terminal of the measurement circuitry 318 is referred to as the reference electrode. FIG. 11 illustrates the locations of the recording and reference electrodes in the six candidate MECs according to one implementation of the present technology suitable for an electrode array 150 configured as two parallel percutaneous leads. Each candidate MEC is represented in one row of the table 1100 beneath a graphical representation 1110 of a twelve-contact lead. The electrodes labelled Rec and Ref in each row are the recording and reference electrodes in the corresponding MEC. The electrodes labelled S and R are the stimulus and return electrodes of a tripolar SEC located, as described above, at one end of the lead.

In an alternative implementation of the present technology, the APM is provided with the patient's selected SECs by a means other than the stages 1060 to 1070. In such an implementation, the APM implements an “ECAP-only” workflow, described below.

### Patient-Controlled Stimulus Ramp (PCSR) Stage

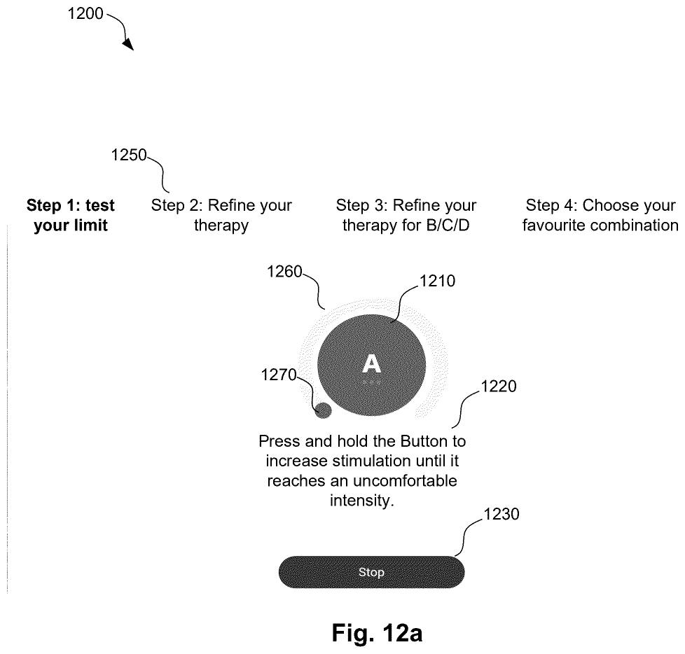

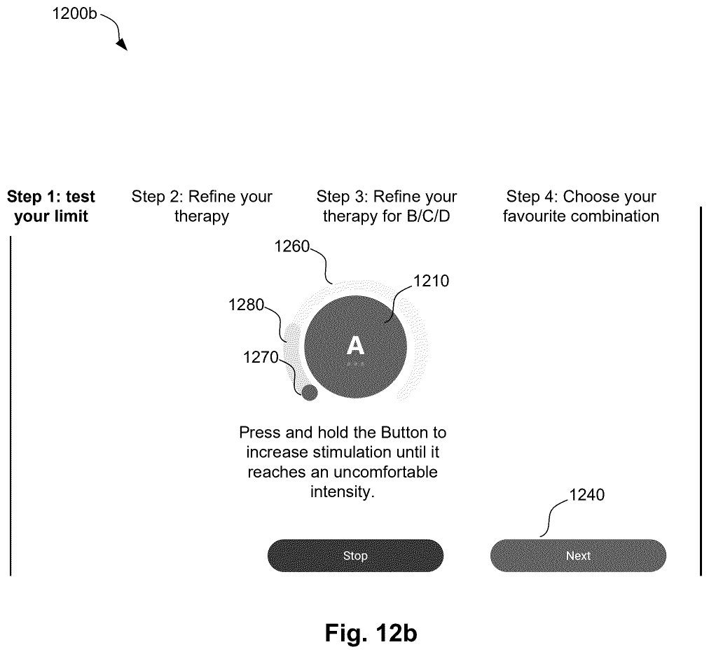

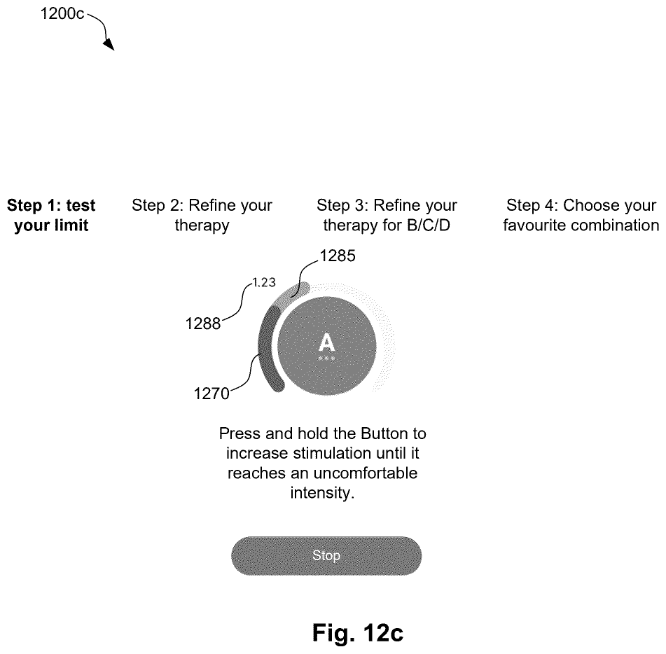

In one implementation of the PCSR stage 1060, the APM renders on the UI display of the CI 740 a screen 1200 as illustrated in FIG. 12a. The screen 1200 comprises a stimulation control 1210 (illustrated as a virtual button), a set of instructions 1220, a Stop control 1230, and a progress bar 1250. The stimulation control 1210, once enabled, is configured to remain activated as long as the patient continues to interact with it, for example by “holding down” the virtual button. In other implementations of the PCSR stage 1060, the stimulation control 1210 or the Stop control 1230 is a hardware control, such as a button, forming part of the UI of the CI 740 yet remaining separate from the display.

The instructions 1220 are configured to instruct the patient to activate the stimulation control 1210, e.g. by holding down the virtual button. When the stimulation control 1210 is activated, the APM instructs the device 710 to deliver stimulation via the current candidate SEC at a gradually increasing or “ramping” stimulus intensity. The identity of the current candidate SEC is indicated by an identifying indicium, e.g. “A”, within the stimulation control 1210. The appearance of the stimulation control 1210 may change during activation, for example to a more muted shade of fill colour. The screen 1200 may be animated to dynamically indicate the stimulus intensity, for example by an animated filling of a graphical element 1260 adjacent to the stimulation control 1210. In one implementation, the graphical element 1260 is a circumferential bar surrounding the stimulation control 1210 as illustrated in FIG. 12a. In this example, an animated filled section 1270 of the circumferential bar 1260 that represents the stimulus intensity as a proportion of a predetermined maximum stimulus intensity is filled in a different manner to the remainder of the circumferential bar 1260. While the stimulation control 1210 is activated, a ratio of the animated filled section 1270 to an area of the circumferential bar 1260 is maintained at a ratio of the stimulus intensity to the predetermined maximum stimulus intensity. Therefore, the filled section 1270 grows larger in proportion to the stimulus intensity until it encompasses the entire bar 1260, at which point the stimulus intensity equals the maximum stimulus intensity. This animation indicates to the patient that something is happening when they activate the control 1210, even if they don't feel stimulation immediately (due to the stimulus intensity being below the perception threshold). The animation also conveys the rate of increase of stimulus intensity to the patient. The animation also reinforces the instructions 1220. That is, even before the patient is able to feel stimulation, the patient can see the filled section 1270 increasing when they activate the control 1210 and decreasing when they de-activate it.

The APM continues to ramp the stimulus intensity as long as the patient continues to activate the stimulation control 1210. In one implementation of the stimulus ramp, the increase in intensity is linear with time, with a predetermined ramp rate. The predetermined ramp rate may be set to 400 microamps/sec to minimise the risk of uncomfortable stimulation. In other implementations the increase in intensity may be non-linear in time.

The stimulus ramp may in some implementations be aborted due to an error condition such as out-of-compliance. The APF returns an out-of-compliance condition if the pulse generator 124 is unable to deliver the current requested by the APM under the current tissue conditions. In such implementations, if a stimulus ramp is aborted due to an out-of-compliance condition, the SEC is adjusted to increase the out-of-compliance current limit, for example as described in US Patent Publication No. US2024/0173550, the contents of which are herein incorporated by reference. The PCSR stage is then repeated for the adjusted SEC.

When the patient de-activates the stimulation control 1210, e.g. by releasing the virtual button, the APM records the stimulus intensity upon release as the Max value for the current SEC. The APM then ramps down the stimulus intensity. In one implementation, the down-ramp of intensity follows a linear profile, with the rate chosen such that the intensity reaches zero after a predetermined interval, for example three seconds. As the stimulus intensity ramps down, the filled section 1270 grows smaller in proportion to the decreasing stimulus intensity until it disappears when the stimulus intensity reaches zero. In other implementations the down-ramp of intensity may be non-linear in time.

If the Stop control 1230 is activated, the stimulation ceases immediately.

The instructions 1220 encourage the patient to continue to activate the stimulation control 1210 for as long as they can tolerate the increasing stimulus intensity, ceasing the activation only when the intensity of stimulus begins to feel uncomfortable. This user interface design takes advantage of the human withdrawal reflex, whereby the patient is likely to instinctively release the button upon receiving uncomfortable stimulation. The design of the PCSR stage 1060 therefore minimises the training burden placed on the patient in using the APM. If the patient does not cease to activate the stimulation control 1210 before the stimulus intensity reaches the maximum stimulus intensity, the APM ceases the stimulus ramp at the maximum stimulus intensity and begins a down-ramp. The stimulus intensity at the point of ceasing the stimulus ramp is recorded as the patient's Max value for that SEC.

The progress bar 1250 indicates progress through the workflow 1050. In onc implementation, the progress bar 1250 comprises four sections corresponding to four steps, each section accompanied by some text indicating the corresponding step. The section (and text) corresponding to the step currently in progress is highlighted by being rendered in a different style to the other sections. For example, in FIG. 12a the section and text are highlighted to indicate that the patient is currently in step 1 (the PCSR stage).

Before and during each stimulus ramp, the APM collects and analyses data as described below. Following a successful stimulus ramp (as defined below), a Next control 1240 is rendered (see FIG. 12b). On activation of the Next control 1240, the PCSR stage 1060 ends successfully and the workflow 1050 proceeds to the coverage survey stage 1065.

### Data Analysis During PCSR Stage

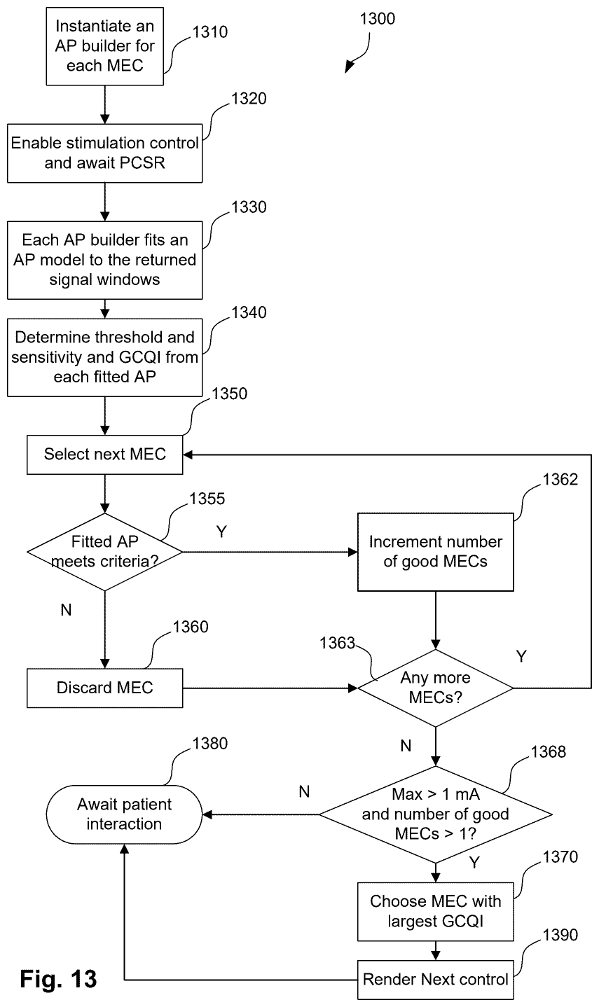

FIG. 13 is a flowchart illustrating a data collection and analysis method 1300 carried out by the APM and the device 710 during the PCSR stage 1060 according to one implementation of the APM. The method 1300 is carried out for each stimulus ramp for each candidate SEC. The method 1300 starts at step 1310. Step 1310 takes place before the APM enables the stimulation control 1210 and therefore before any stimulus is applied.

Step 1310 instantiates, for each candidate MEC in the list of candidate MECs for the current SEC (see FIG. 11), an activation plot (AP) builder. The AP builder is described in more detail below.

The method 1300 then proceeds to step 1320, which enables the stimulation control 1210 and waits for the patient to commence the PCSR for the current SEC as described above. During the PCSR, the APM instructs the device 710 to capture and return signal windows at each candidate MEC for each stimulus intensity parameter value s. The returned signal windows for each candidate MEC are analysed by the corresponding AP builder, which measures a neural response intensity d from each signal window in the same manner as is done by the ECAP detector 320. The AP builder thus accumulates a set of (s, d) value pairs for the corresponding candidate MEC.

The AP builder also counts the number of clipping flags returned by the APF with each signal window. A clipping flag is returned by the APF if a sample in the signal window falls outside a predetermined range of values. If the number of clipping flags throughout the PCSR for any MEC exceeds some fraction of the total number of samples in all signal windows for that MEC, such as 5%, that candidate MEC is excluded from all further processing and its signal windows discarded.

Once the stimulation control 1210 is de-activated, at step 1330, each AP builder fits an activation plot to the set of (s, d) value pairs for the corresponding candidate MEC. Each AP builder then at step 1340 determines an ECAP threshold and a patient sensitivity for its fitted activation plot and calculates a growth curve quality index (GCQI) for the fitted activation plot. Activation plot fitting and the calculation of the GCQI by the AP builder are described in more detail below.

Step 1350 then selects one of the candidate MECs for the current SEC. Step 1355 then tests whether the fitted activation plot for the current candidate MEC meets certain inclusion criteria. The purpose of the inclusion criteria of step 1355 is to confirm that the fitted activation plot can be trusted. The inclusion criteria are:
The GCQI is greater than a threshold, e.g. 10 dB.
The ECAP threshold determined from the activation plot at step 1340 is within a predetermined range. In one implementation, the range is from the first percentile to the 99th percentile of the distribution of ECAP thresholds obtained from existing patient data.
The patient sensitivity determined from the activation plot at step 1340 is within a predetermined range. In one implementation, the range is from the first percentile to the 99th percentile of the distribution of patient sensitivities obtained from existing patient data.
The standard error of the determined patient sensitivity is less than a threshold, e.g. 0.5 times the determined sensitivity.

If the fitted activation plot does not meet the inclusion criteria (“N” at step 1355), the method 1300 at step 1360 discards the current candidate MEC, then at step 1363 determines whether there are any remaining candidate MECs. If so (“Y”), the method 1300 returns to step 1350 to select the next candidate MEC.

If the fitted activation plot does meet the inclusion criteria (“Y” at step 1355), the current MEC is deemed “good”. Step 1362 increments the number of “good” MECs (which was initialised to zero at the start of the method 1300), and the method 1300 proceeds to step 1363.

Once all the candidate MECs have been exhausted (“N” at step 1363), step 1368 tests whether the number of “good” MECs is greater than one, and the Max value is greater than a threshold, e.g. 1 mA. If so (“Y”), the method 1300 at step 1370 selects one of the “good” MECs for the current candidate SEC. In one implementation, the selected MEC is the MEC with the highest GCQI. The method 1300 then renders the Next control 1240 at step 1390, and proceeds to step 1380 to wait for a patient interaction. If not (“N” at step 1368), the method 1300 proceeds directly to step 1380 without rendering the Next control 1240.

The patient may activate the Stop control 1230 to end the method 1300 at any time. If the method 1300 ends in this fashion, the PCSR stage 1060 ends with the current candidate SEC marked as unsuccessful, meaning it takes no further part in the workflow 1050. As mentioned above, activation of the Next control 1240 once step 1380 is reached ends the PCSR stage 1060 successfully for the current SEC. Alternatively, the patient may choose to repeat the method 1300 for the current candidate SEC by activating the stimulation control 1210 once step 1380 is reached. There may be no limit to the number of times the method 1300 may be repeated for a current candidate SEC. Alternatively, a limit on the number of iterations of the PCSR stage for a candidate SEC may be imposed. Once that limit is reached, the PCSR stage 1060 ends with the current candidate SEC marked as unsuccessful.

If the method 1300 is iterated a second or subsequent time for a candidate SEC, the PCSR screen may appear as illustrated in FIG. 12b. In the screen 1200b illustrated in FIG. 12b, the circumferential bar 1260 contains two filled sections: the animated filled section 1270 of darker huc, dynamically indicating the current stimulus intensity as described above, and a residual filled section 1280 of lighter hue statically indicating the Max value for that SEC, as a proportion of the maximum stimulus intensity. The residual filled section 1280 encourages the patient to achieve or surpass their previous Max value. The residual filled section 1280 also provides information to a clinician supervising the workflow about the result of the patient's previous PCSR iterations for a candidate SEC. The screen 1200b also contains a Next control 1240, although this will not necessarily be present during a second or subsequent iteration.

In some implementations, on a second or subsequent iteration of the method 1300 for a candidate SEC, the circumferential bar 1260 may contain a second residual filled section, statically indicating a “target” stimulus intensity that would achieve a target Normalised Dose Ratio (see below) for the candidate SEC, as a proportion of the maximum stimulus intensity. The second residual filled section 1285, as illustrated on the PCSR screen 1200c in FIG. 12c, may be of different appearance from both the animated filled section 1270 and the first residual filled section 1280 (FIG. 12b). In such an implementation, the PCSR screen 1200c may also contain an indicator 1288 of the target dose ratio (equal to 1.23 in FIG. 12c) represented by the second residual filled section 1285. In one such implementation, once the stimulus intensity reaches the target stimulus intensity, the PCSR ceases and the stimulus intensity ramps back down to zero, independently of whether the patient continues to activate the stimulation control 1210.

In some implementations, the circumferential bar 1260 may contain a third residual filled section, statically indicating the ECAP threshold as a proportion of the maximum stimulus intensity. Such a third residual filled section may be displayed in conjunction with, or independently of, the second residual filled section 1285. The third residual filled section may be of different appearance from the animated filled section 1270, the first residual filled section 1280 (FIG. 12b), and (if present) the second residual filled section 1285.

The result of the PCSR stage 1060 as implemented by the method 1300 is a candidate SEC marked as successful or unsuccessful. Each successful candidate SEC is also accompanied by a Max value, an MEC chosen at step 1370, a corresponding activation plot, and a GCQI value for the activation plot. The activation plot is defined by its parameters, from which patient characteristics such as ECAP threshold and patient sensitivity may be determined, as described below.

If there are no successful candidate SECs after the method 1300 has been carried out at least once for each candidate SEC, the workflow 1050 ends unsuccessfully.

### Coverage Survey Stage

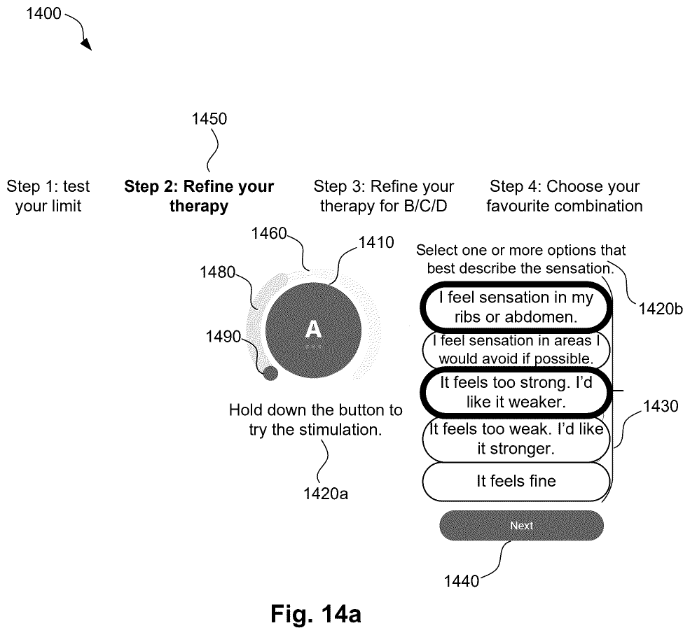

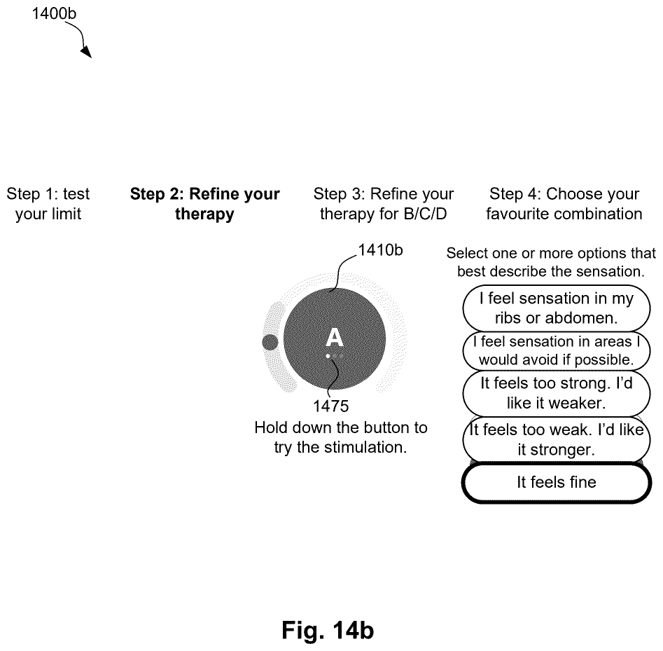

In one implementation of the Coverage Survey stage 1065, the APM renders on the UI display of the CI 740 a screen 1400 as illustrated in FIG. 14a. The screen 1400 comprises a stimulation control 1410, a set of instructions 1420a and 1420b, a set of options 1430, a Next control 1440, and a progress bar 1450. In other implementations of the Coverage Survey stage 1065, the stimulation control 1410 or the Next control 1440 are hardware controls, such as buttons, forming part of the UI of the CI 740 yet remaining separate from the display.

The screen 1400 is rendered at least once for each successful candidate SEC from the PCSR stage 1060 to assess that candidate SEC. The stimulation control 1410 is in the form of a control such as a virtual button that, like the stimulation control 1210, is configured to remain activated as long as the patient continues to interact with it, for example by “holding down” the virtual button. When the stimulation control 1410 is activated, the APM instructs the device 710 to deliver stimulation via the current candidate SEC. The identity of the current candidate SEC is indicated by an identifying indicium, e.g. “A”, within the stimulation control 1410. The appearance of the stimulation control 1410 may change during activation, for example to a more muted shade of fill colour. In one implementation of the coverage survey stage 1065, stimulation turns on and off at the current candidate SEC by threshold ramps to and from a comfortable stimulus intensity for the current candidate SEC. The threshold for the threshold ramp may be the ECAP threshold for the current candidate SEC, as determined at the PCSR stage 1060. Threshold ramps are described below.

An initial comfortable stimulus intensity for each candidate SEC may be predicted at the start of the coverage survey stage 1065 for that candidate SEC from the Max value sₘₐₓ and the ECAP threshold s_{T} that were estimated and determined for the candidate SEC at the PCSR stage 1060. In one implementation, the comfortable stimulus intensity s_{comf} may be calculated as a fixed proportion of the interval between s_{T} and sₘₐₓ for the candidate SEC:

$${s}_{c o m f} = {s}_{T} + k ( {s}_{max} - {s}_{T} ) \qquad ( 6 )$$

where k is a predetermined constant between 0 and 1. In an alternative implementation, the comfortable stimulus intensity s_{comf} may be calculated as follows:

$${s}_{c o m f} = {s}_{T} + k ( {s}_{max} - {s}_{T} ) + c \cdot log ( S ) + d \qquad ( 7 )$$

where S is the patient sensitivity that was determined for the candidate SEC at the PCSR stage 1060, and c and d are constants, for example with values −173 and −523 respectively. However, if Equation (7) should result in a value of s_{comf} that exceeds sₘₐₓ or is less than s_{T}, then Equation (6) may be used to calculate s_{comf} instead.

A circumferential bar 1460 may be displayed around the stimulation control 1410. The circumferential bar 1460 may have a residual filled section 1480 to indicate the Max value for the current candidate SEC, as a proportion of the maximum stimulus intensity. On activation of the stimulation control 1410, an element 1490 may be animated within the circumferential bar 1460, such as by traversing around the circumferential bar 1460, to dynamically indicate the progress of the stimulus intensity during the ramp towards the comfortable stimulus intensity for that SEC. The position of the element 1490 within the circumferential bar 1460 is indicative of the current stimulus intensity as a proportion of the maximum stimulus intensity. The current stimulus intensity in relation to the Max value, which is indicated by the residual filled section 1480, may therefore be easily perceived by a user.

The instructions 1420a and 1420b instruct the patient to activate the stimulation control 1410 and to select one or more of the options 1430 to provide feedback about their sensations. Each option 1430 corresponds to a selectable control. The APM then waits for the patient to select one or more of the options 1430 and activate the Next control 1440. The Next control 1440 is disabled until stimulation has been tested and least one option is selected. An option may be toggled between selected and deselected by activating (e.g. touching) the control underlying its text.

In some implementations, for each candidate SEC, the options 1430 are not displayed until after the patient has activated the stimulation control 1410 corresponding to that SEC.

The progress bar 1450 at the top of the screen 1400, like the progress bar 1250, indicates progress through the workflow 1050. In the screen 1400, the leftmost-but-one section and text of the progress bar 1450 are highlighted to indicate that the patient is currently in step 2 (the coverage survey stage) of the workflow 1050.

Once the Next control 1440 is activated, the APM responds to the options selected for the current candidate SEC with a “mitigation” selected according to Table 1. A “1” in a column of Table I represents the selection of the option or options corresponding to that column, a “0” represents non-selection, and an “X” means either the option was selected or not (the selection of the option does not affect the chosen mitigation).

TABLE 1
Mitigations in first iteration of
Coverage Survey for a candidate SEC

| | Third | Fourth | Fifth | |
|---|---|---|---|---|
| First or second | option | option | option | |
| option | (“Too | (“Too | (“Feels | |
| (“discomfort”) | strong”) | weak”) | fine”) | Mitigation |
| 0 | 0 | 0 | 0 | N/A |
| 0 | 0 | 0 | 1 | None |
| 0 | 0 | 1 | X | Increase comfortable |
| | | | | stimulus intensity |
| 0 | 1 | X | X | Decrease comfortable |
| | | | | stimulus intensity |
| 1 | X | X | X | Adjust candidate SEC to a |
| | | | | new location |

The mitigations to increase and decrease the comfortable stimulus intensity do so by a small amount, equal to 0.05×(sₘₐₓ−s_{T}) in one implementation. However, the decrease and increase mitigations are not permitted to move the comfortable stimulus intensity outside the therapeutic range defined as [sₘₐₓ, s_{T}]. If the comfortable stimulus intensity is adjusted according to these mitigations, the Coverage Survey stage 1065 may then be repeated for the adjusted comfortable stimulus intensity.

The “discomfort” mitigation to adjust the current candidate SEC moves the candidate SEC by one or more electrodes towards the middle of the lead. For example, the current candidate SEC may be moved by three electrodes towards the middle of the lead. If the current candidate SEC is moved according to this mitigation, the workflow 1050 returns to the PCSR stage 1060 (described above) for the adjusted candidate SEC. The Coverage Survey stage 1065 is then repeated for the adjusted candidate SEC, if the PCSR stage 1060 was successful for the adjusted candidate SEC.

In some implementations, for each candidate SEC, the “too weak” or the “feels fine” options are not enabled until the control 1410 has been activated for long enough for the stimulation intensity to ramp up to the comfortable stimulus intensity. This prevents the patient from responding to the Coverage Survey stage 1065 with incomplete information.

In some implementations, the first option (“ribs or abdomen”) is not enabled for candidate SECs at the caudal end (bottom) of the lead.

If the Coverage Survey stage 1065 is repeated for an adjusted candidate SEC, the stimulation control 1410 is rendered differently from its original appearance in the first iteration. For example, in the coverage survey screen 1400b as illustrated in FIG. 14b, the leftmost indicium 1475 of the three indicia below the identifying indicium “A” within the stimulation control 1410b is highlighted. This is distinct from the stimulation control 1410 illustrated in FIG. 14A in which none of the three indicia below the identifying indicium “A” within the stimulation control 1410 is highlighted.

In a repeat iteration of the coverage survey stage 1065 for an adjusted candidate SEC, the APM responds to selections for that candidate SEC with a mitigation selected according to Table 2. As in Table 1, a “1” in a column of Table 2 represents the selection of the option corresponding to that column, a “0” represents non-selection, and an “X” means either the option was selected or not (the selection of the option does not affect the chosen mitigation).

TABLE 2
Mitigations in second iteration of
Coverage Survey for a candidate SEC

| | Third | Fourth | Fifth | |
|---|---|---|---|---|
| First or second | option | option | option | |
| option | (“Too | (“Too | (“Feels | |
| (“discomfort”) | strong”) | weak”) | fine”) | Mitigation |
| 0 | 0 | 0 | 0 | N/A |
| 0 | 0 | 0 | 1 | None |
| 0 | 0 | 1 | X | Increase comfortable |
| | | | | stimulus intensity |
| 0 | 1 | X | X | Decrease comfortable |
| | | | | stimulus intensity |
| 1 | X | X | X | Decrease comfortable |
| | | | | stimulus intensity |

In some implementations of the workflow 1050, the PCSR stage 1060 may only be repeated once (i.e. iterated twice) for any candidate SEC, to reduce the burden on the patient of repeatedly having to undergo the PCSR stage 1060 with adjusted SECs.

If the patient still feels sensation in ribs or abdomen or other undesirable areas (first or second options) for a candidate SEC at the second iteration of the Coverage Survey stage 1065 for that candidate SEC, the comfortable stimulus intensity for that candidate SEC is decreased (as per the final row of Table 2). The patient will have the opportunity to select or discard that candidate SEC during the Coverage Selection stage 1070.

In an alternative implementation, that candidate SEC is marked as unsuccessful. The Coverage Survey stage 1065 is not repeated for that candidate SEC. The patient will not have the opportunity to select that candidate SEC during the Coverage Selection stage 1070.

The final Coverage Survey stage 1065 ends with a set of successful candidate SEC/MEC combinations and their respective comfortable stimulus intensities.

### AP Builder

As mentioned above, the AP builder, as used for example at step 1330 of the method 1300, fits an activation plot using a model referred to as the Golden Growth Curve (GGC) to a set of (s, d) value pairs, where d is a measured neural response intensity from a signal window and s is the corresponding stimulus intensity parameter. The AP builder may also, for example at step 1340 of the method 1300, calculate a growth curve quality index (GCQI) for a fitted activation plot.

An important part of the AP builder is an ECAP detector that returns the neural response intensity (e.g. the ECAP amplitude) d from a signal window. In one implementation, the ECAP detector described in the International Patent Publication no. WO2024/065013 by the present applicant, the contents of which are herein incorporated by reference, may be used by the AP builder to measure the amplitude d of the ECAP in a signal window. Alternatively, the ECAP detector described in the above-mentioned International Patent Publication no. WO2015/074121 may be used by the AP builder to measure the amplitude d of the ECAP in a signal window. In the latter case, the ECAP detector has two parameters: its correlation delay, and its length (or equivalently its frequency). Other implementations of ECAP detectors may have other adjustable parameters. The optimal values of these parameters are dependent on the SEC and the MEC that gave rise to the signal window and should therefore be customised for each instance of the AP builder, for example as instantiated at step 1310 of the method 1300. In one implementation, the AP builder may customise the ECAP detector parameters on an average signal window obtained by averaging the ten signal windows corresponding to the largest values of stimulus intensity s. In one implementation, an ECAP presence classifier may first be applied to each signal window before incorporating it into the average signal window. If the ECAP presence classifier indicates that the signal window did not contain a neural response, the signal window is discarded. One example of an ECAP presence classifier is the Noise Departure Detector described in International Patent Publication no. WO2023/115157 by the present applicant, the contents of which are herein incorporated by reference.

In one example of customising an ECAP detector for a given SEC/MEC combination, the ECAP detector described in the above-mentioned International Patent Publication no. WO2024/065013 may be derived by projecting the average signal window onto an artefact basis, and subtracting the projection from the average signal window to obtain a residual that is by definition orthogonal to the artefact basis, as described in the above-mentioned International Patent Publication no. WO2024/065013. The normalised residual is the ECAP detector that is customised for the SEC/MEC combination.

Once the ECAP detector has been customised and the set of (s, d) value pairs has been obtained, the AP builder proceeds to fit a GGC model to the set of (s, d) value pairs. Like the piecewise linear model of equation (1), the GGC model is a continuous model comprising a sub-threshold linear portion of constant zero intensity and a supra-threshold linear portion. However, in the GGC model, these two portions are joined by a transitional portion of variable curvature. In one implementation, the GGC model is derived from a multi-parameter template function g(x|τ, x₀) with two such linear portions and a curved transitional portion. The parameters of the template function g are:
x₀, the x-intercept of the supra-threshold linear portion and therefore the approximate location of the transitional portion;
τ, a curvature parameter that sets the curvature of the transitional portion (the smaller the value of curvature t, the sharper the transition).

One implementation of the GGC model is the difference between two different versions of the template function g having different transitional locations and curvatures but the same scaling:

$$d ( s ) = P T [ g ( \frac{s}{T} \vert {τ}_{1} , 1 ) - g ( \frac{s}{T} \vert {τ}_{2} , r ) ] \qquad ( 8 )$$

This implementation, referred to as the bounded GGC model, comprises three distinct portions: a sub-threshold portion of zero intensity, a supra-threshold linear portion joined to the sub-threshold portion by a first transitional portion around an x-intercept, and a saturation portion that approaches a saturation value joined to the supra-threshold portion by a second transitional portion around a saturation threshold. The parameters of such an implementation of the GGC model are:
P, the slope of the supra-threshold linear portion, related to the patient sensitivity
T, the x-intercept of the supra-threshold linear portion, related to the ECAP threshold
τ₁, the curvature parameter of the first transitional portion
τ₂, the curvature parameter of the second transitional portion
r, the ratio of the saturation threshold to the intercept T.

In other implementations, more parameters may be used for the GGC model, for example a GGC model in which the sub-threshold portion has a non-zero intensity such as a constant intensity (one parameter) or a linear profile (two parameters), to model the effect of any artefact that leaks through the ECAP detector.

To fit the GGC, the parameters P, T, τ₁, τ₂, and r may be initialised to sensible respective starting points P₀, T₀, τ₀, τ₀, and r₀. In one implementation, these values may be set to:
P₀: the slope of a linear regression applied to the subset of data with stimulus intensity parameter values greater than TO
T₀: the maximum stimulus intensity parameter value in the set of value pairs
τ₀: between 0.1 and 0.4
r₀: between 3 and 4

A fitting algorithm such as Trust Region Reflective (TRF) may then be used to optimise the values of the parameters P, T, τ₁, τ₂, and r from the starting points P₀, T₀, τ₀, and r₀. Alternatively, a hybrid approach may be used in which iterations of TRF to optimise the nonlinear parameters T, τ₁, τ₂, and r are interleaved with iterations of ordinary least squares to optimise the linear parameter P.

In some implementations, the optimisation algorithm returns a standard error (e.g. a standard deviation) for each parameter as well as a value for the parameter itself.

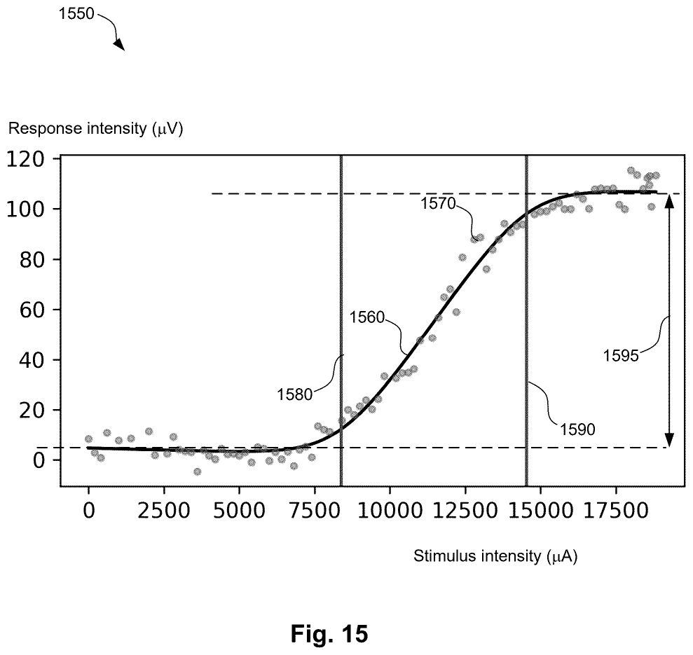

FIG. 15 is a graph 1550 containing a bounded GGC model 1560 fitted to a set of (stimulus intensity, response intensity) value pairs (e.g. the pair 1570). It may be seen that the fitted GGC 1560 shows some saturation at higher stimulus intensities, i.e. at stimulus intensities above the saturation threshold sₛₐₜ. The vertical line 1580 represents the intercept T and the vertical line 1590 represents the saturation threshold Isut (the ratio r times the intercept T).

The AP builder may also, for example at step 1340 of the method 1300, calculate a growth curve quality index (GCQI) for the fitted GGC model. The GCQI indicates a signal-to-noise ratio (SNR) of the fitted GGC. In one implementation, the AP builder may calculate the GCQI by dividing the peak-to-peak amplitude of the fitted GGC by the standard deviation of the residuals of the fitted GGC. The peak-to-peak amplitude of a bounded GGC (e.g. as indicated in FIG. 15b by the arrow 1595) may be determined as the difference between the response intensity at the ECAP threshold s_{T} and the response intensity at the saturation threshold sₛₐₜ.

The fitted GGC may be used to determine the ECAP threshold s_{T}, as in step 1340 of the method 1300. In one implementation, the ECAP threshold s_{T} is the intercept T.

The fitted GGC may also be used to determine the patient sensitivity S, as in as in step 1340 of the method 1300. In one implementation, the patient sensitivity S is the slope P. The standard error of the patient sensitivity S (part of the inclusion criteria used at step 1355) is the standard error of the slope P returned by the fitting algorithm.

### Threshold Ramp

A threshold ramp is a ramp of stimulus intensity, either up or down, that traverses stimulus intensity values below a predetermined threshold value at a faster rate than the ramp traverses stimulus intensity values above the predetermined threshold value.

When ramping stimulus intensity up, it is preferred by patients that the ramp feel gradual rather than abrupt. However, it is also generally desirable to produce a user interface that feels responsive to the patient. For example, during the PCSR stage 1060, the patient may de-activate the stimulation control 1210, causing the stimulation to turn off. If they do so in response to an uncomfortable stimulus, the responsiveness of the user interface is important. A patient will be more willing to experiment with their comfort limits if stimulation ramps down quickly without producing discomfort.

Stimulus intensities below the ECAP threshold are generally not perceivable by patients. Therefore, ramping through sub-ECAP-threshold intensities does not improve the patient's sensation of gradualness and may in fact detract, by taking up unnecessary time, from the patient's sensation of responsiveness. A threshold ramp may therefore skip over most sub-ECAP-threshold stimulus intensities on either the way up or the way down.

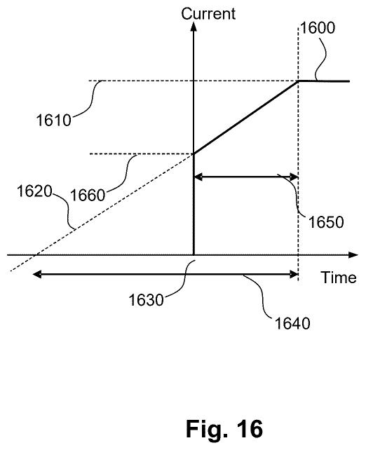

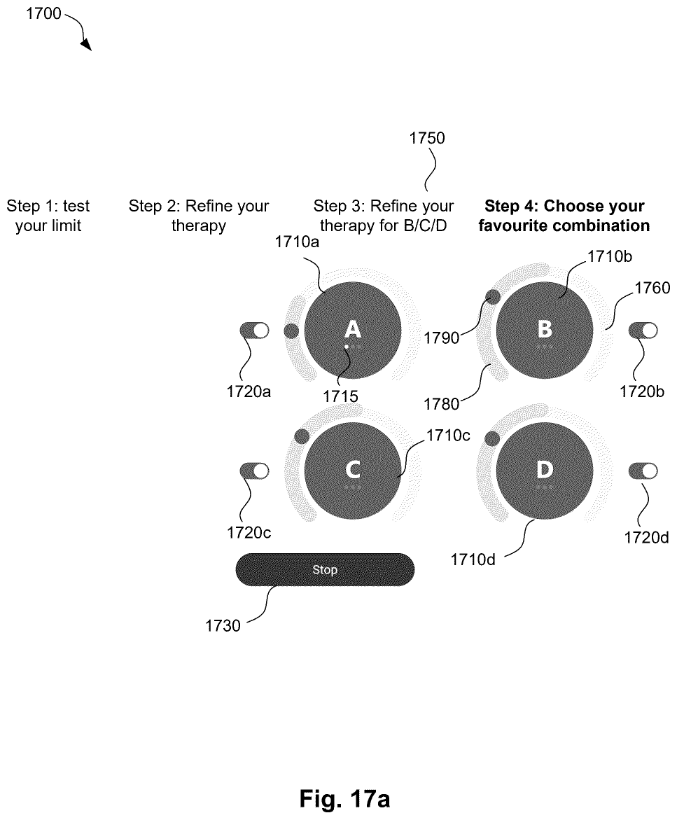

FIG. 16 illustrates a threshold ramp according to one implementation of the present technology. The profile 1600 represents the time course of stimulus current amplitude according to a threshold ramp up to a target current amplitude 1610. The dotted profile 1620 represents the time course of stimulus current amplitude according to a conventional linear ramp from zero to the target current amplitude 1610. The instant 1630 represents the time (t=0) at which the ramp was initiated, e.g. by activation of the stimulation control 1210. The interval 1640 represents the predetermined time that would have been taken by the conventional linear ramp, for example three seconds, to reach the target current amplitude 1610. The ramp rate of the conventional linear ramp profile 1620 is calculated such that the stimulus intensity reaches the target current amplitude 1610 at the end of the interval 1640. The threshold ramp, by contrast, steps comparatively rapidly (e.g. vertically) to a threshold current amplitude 1660. Then during the interval 1650, the threshold ramp linearly increases the stimulus current amplitude at the same rate as the conventional linear ramp. The length of the interval 1650, i.e. the total ramp time, is therefore significantly less than the predetermined time of the interval 1640. The threshold ramp therefore appears more responsive to the patient. Moreover, if the threshold current amplitude 1660 is set slightly below the ECAP threshold, the threshold ramp does not appear any more abrupt than the conventional linear ramp, since the patient is unable to perceive stimulus current amplitudes below the threshold current amplitude 1660.

In one implementation, the threshold current amplitude 1660 may be obtained by scaling the ECAP threshold by 0.9. This scaling factor provides a balance between having faster overall ramp times and keeping the likelihood of a step to a perceptible current amplitude low.

A threshold down-ramp according to one implementation is a time-reversed version of the profile 1600 of the threshold ramp illustrated in FIG. 16. In other words, a threshold down-ramp from a starting current amplitude decreases current amplitude linearly at a rate equivalent to a conventional linear down-ramp over the predetermined interval 1640. When the stimulus current amplitude reaches the threshold current amplitude 1660, the stimulus current amplitude steps comparatively rapidly (e.g. vertically) to zero.

In other implementations of the threshold ramp, the profile of stimulus current amplitude is not piecewise linear as in FIG. 16. Instead, alternative profiles of stimulus intensity may be used. The alternative profiles are also parametrised by a threshold value. In one such implementation, the profile follows a sigmoid function, such as described above, that smoothly and exponentially rises from zero to a midpoint that is computed from the threshold, and decelerates as the stimulus current amplitude approaches the target current amplitude. Another such implementation is an exponential profile below the threshold, followed by a linear profile above the threshold. The ramp rate of the linear profile is chosen to be less than the average ramp rate of the exponential profile.

In some implementations, as described above in relation to the Patient-Controlled Stimulus Ramp stage 1060, a threshold ramp may be interrupted if the APF receives no communication from the APA within a first timeout period. The controller 116 may then ramp the intensity back down in the continued absence of communication from the APA within a second timeout period. In such implementations, the patient is less likely to receive uncomfortable stimulation if the communication between the APF and the APM is interrupted.

### Coverage Selection Stage

As mentioned above, the coverage selection stage 1070 is configured to receive input from the patient to select one or more of the successful candidate SECs from the coverage survey stage 1065. The coverage selection stage 1070 allows the patient to test different combinations of candidate SECs before selecting which ones to keep.

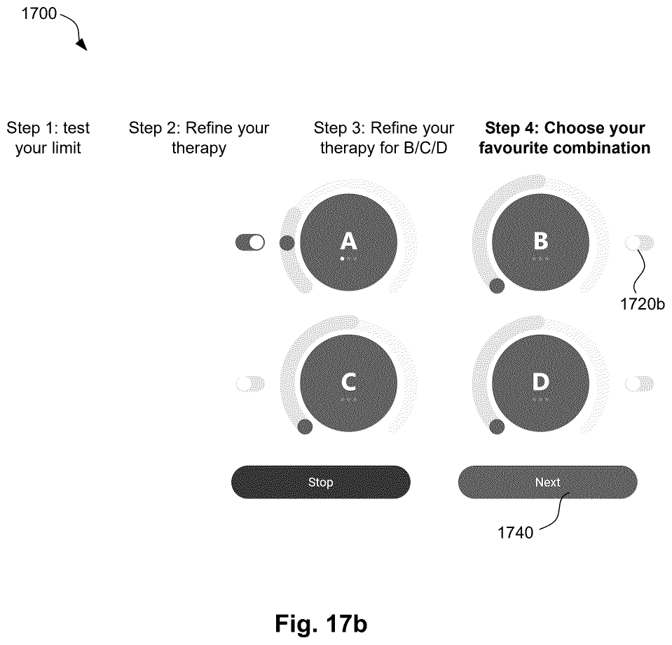

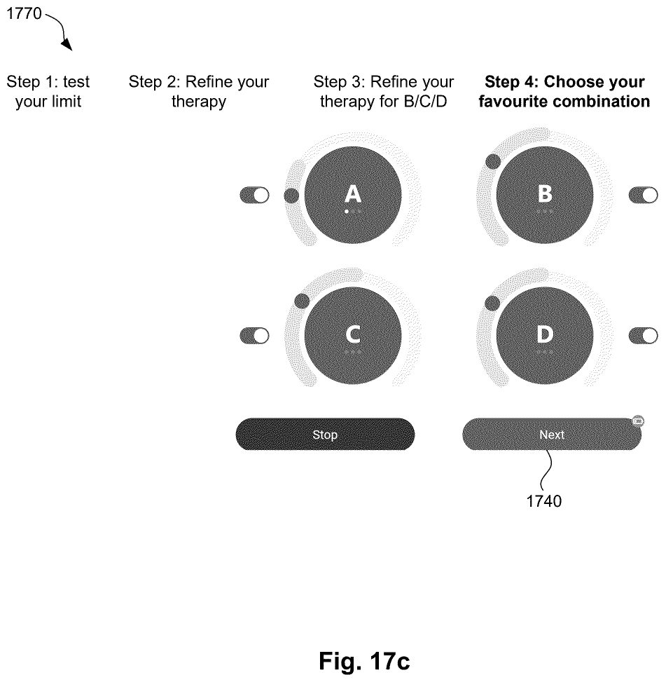

In one implementation of the coverage selection stage 1070, the APM renders on the UI display of the CI 740 a screen 1700 as illustrated in FIGS. 17a and 17b. The coverage selection screen 1700 comprises controls comprising: up to four tiles, e.g. 1710a, 1710b, 1710c, and 1710d; up to four respective toggle switches, e.g. 1720a, 1720b, 1720c, and 1720d; a Next control 1740 (FIG. 17b); a progress bar 1750; and a Stop control 1730.

Toggle switches 1720a, 1720b, 1720c, and 1720d are associated with respective tiles 1710a, 1710b, 1710c, and 1710d to form control pairs. In some implementations, the switch corresponding to a tile is not rendered until the tile has been activated once for a predetermined minimum duration, e.g. five seconds. In the coverage selection screen 1700 illustrated in FIG. 17a, tiles 1710a, 1710b, 1710c, and 1710d have all been activated, so tiles 1710a, 1710b, 1710c, and 1710d have associated switches 1720a, 1720b, 1720c, and 1720d respectively.

In other implementations of the coverage selection stage 1070, one or more of the controls are hardware controls, such as buttons or switches, forming part of the UI of the CI 740 yet remaining separate from the display.

Each control pair, e.g. the tile 1710b and the switch 1720b, corresponds to one of the successful candidate SECs after the coverage survey stage 1065. The identity of the candidate SEC corresponding to a tile is indicated by an identifying indicium, e.g. “A”, within the tile. In some implementations, the toggle control pairs are physiologically ordered. That is to say, the position in which each toggle control pair appears on the coverage selection screen 1700 corresponds to the physical position of its corresponding SEC on the electrode array 150, and therefore to the relative location on the body where paresthesia induced by stimulation controlled by that control pair may be felt. In one such implementation, as illustrated in FIG. 17a, suitable for the situation described above in which the four candidate SECs are defined as top left, top right, bottom left, and bottom right of a pair of parallel percutaneous leads, the control pair (1710a, 1720a) corresponds to candidate SEC “A”, the control pair (1710b, 1720b) corresponds to candidate SEC “B”, the control pair (1710c, 1720c) corresponds to candidate SEC “C”, and the control pair (1710d, 1720d) corresponds to candidate SEC “D”. Physiological ordering may assist the patient in recalling the effect of each control pair without having to interact with it, and therefore may contribute to a more efficient coverage selection stage 1070.

As in the coverage survey screen 1600, a tile corresponding to a candidate SEC that was adjusted during the coverage survey stage 1065 may be rendered differently to tiles corresponding to unadjusted candidate SECs. For example, in the coverage selection screen 1700 as illustrated in FIG. 17a, the leftmost indicium 1715 of the three indicia below the identifying indicium “A” within the tile 1710a is highlighted, by contrast with the tile 1710b in which none of the three indicia below the identifying indicium “B” is highlighted. In this way, the coverage selection screen 1700 may indicate to a user which of the corresponding candidate SECs has been adjusted during the coverage survey stage 1065.

The (tile, switch) control pairs may be activated and de-activated independently. Each tile is configured to remain activated as long as the patient continues to interact with it, for example by “holding down” the tile, and becomes de-activated when the patient ceases to interact with it, for example by “releasing” the tile. Like the stimulation controls 1210 and 1410, the tile may take on a different appearance when it is activated, for example by being filled in more muted colour. By contrast, each toggle switch cannot be “held down”, but inverts its state from de-activated to activated or from activated to de-activated each time the patient interacts with the toggle switch, for example by “tapping” the toggle switch.

The state of stimulation on an SEC (active or inactive) corresponds to the state of the corresponding toggle switch (activated or de-activated). That is, the stimulation is always on if the switch is activated, and always off if the switch is de-activated. The state of a toggle switch therefore offers a visual cue to indicate the state of stimulation on the corresponding SEC. This may be emphasised in some implementations by rendering the toggle switch differently depending on whether it is activated or de-activated, for example by filling the switch in a more muted colour when it is de-activated, as illustrated for the de-activated switch 1720b in FIG. 17b.

The stimulus pulses from all the active SECs at a given time are delivered interleaved, staggered in time by a predetermined inter-stimulus interval, as described above in relation to FIG. 8. In one implementation, a stimulus pulse from SEC “A” is delivered first, followed by SEC “B”, then SEC “C”, then SEC “D”. In one implementation, the inter-stimulus interval is the stimulus period divided by the number of successful candidate SECs, so that the stimuli are evenly spaced throughout the stimulus period.

In one implementation, the tiles have an inverting behaviour, whereby for as long as the tile is being activated, e.g. held down, the state of stimulation, which is always indicated by the state of the toggle switch, is inverted. For example, if a toggle switch is activated, activating the corresponding tile de-activates the toggle switch and stops stimulation, and de-activating the tile activates the toggle switch and restarts stimulation. Conversely, if a toggle switch is de-activated, activating the corresponding tile activates the toggle switch and starts stimulation, and de-activating the tile de-activates the toggle switch and stops stimulation.

Table 3 summarises the effect of activating and de-activating the tile and the toggle switch on the stimulation from the corresponding candidate SEC according to this implementation of the coverage selection stage 1070. Blank cells represent actions that cannot occur.

TABLE 3
State transition table for one implementation
of coverage selection stage

| State | Stimulation / | Activate | De-activate | Activate | De-Activate |
|---|---|---|---|---|---|
| No. | Switch state | tile | tile | switch | switch |
| 1 | Off / | 2 | 2 | 2 | |
| | De-activated | | | | |
| 2 | On / | 1 | 1 | | 1 |
| | Activated | | | | |

In another implementation, if the toggle switch is activated, activating the corresponding tile de-activates the toggle switch and stops stimulation, and de-activating the tile does not further change the state of stimulation. Conversely, if the toggle switch is de-activated, activating the corresponding tile activates the toggle switch and starts stimulation, and de-activating the tile de-activates the toggle switch and stops stimulation. Table 4 summarises the effect of activating and de-activating the tile and the toggle switch on the stimulation from the corresponding candidate SEC according to this implementation of the coverage selection stage 1070.

TABLE 4
State transition table for alternative implementation
of coverage selection stage

| | Stimulation / | Activate | De-activate | Activate | De-Activate |
|---|---|---|---|---|---|
| State No. | Switch state | tile | tile | switch | switch |
| 1 | Off / | 2 | 1 | 2 | |
| | De-activated | | | | |
| 2 | On / | 1 | 1 | | 1 |
| | Activated | | | | |

Under the implementation summarised in Table 4, the behaviour of stopping stimulation when a stimulus control is de-activated, as during the PCSR and coverage survey stages, is maintained.

Yet a further implementation is a hybrid of Table 3 and Table 4 depending on how long the tile remains activated. Specifically, if a toggle switch is activated, activating the corresponding tile de-activates the toggle switch and stops stimulation, and de-activating the tile after less than a predetermined interval activates the toggle switch and restarts stimulation. However, de-activating the tile after greater than the predetermined interval does not further change the state of stimulation, i.e. the switch remains de-activated and stimulation remains off.

Conversely, if the toggle switch is de-activated, activating the corresponding tile activates the toggle switch and starts stimulation, and de-activating the tile after less than the predetermined interval de-activates the toggle switch and stops stimulation. However, de-activating the tile after greater than the predetermined interval does not further change the state of stimulation, i.e. the switch remains activated and stimulation remains on.

The progress bar 1750, like the progress bars 1250 and 1450, indicates progress through the workflow 1050. In FIG. 17a, the rightmost section and text of the progress bar 1750 are highlighted to indicate that the patient is currently in step 4 (the coverage selection stage 1070) of the workflow 1050.

The Stop control 1730 disables all stimulation and de-activates all toggle switches 1720a, 1720b etc.

In an alternative implementation of the coverage selection stage 1070, there are no tiles, only toggle switches.

In one implementation of the coverage selection stage 1070, stimulation turns on and off at a candidate SEC by threshold ramps to and from the comfortable stimulus intensity for the candidate SEC that resulted from the Coverage Survey stage 1065. The threshold for the threshold ramp is the ECAP threshold for the candidate SEC that was estimated at the PCSR stage 1060. Threshold ramps are described above.

As in the coverage survey screen 1400, a circumferential bar may be displayed around each tile, e.g. the circumferential bar 1760 around the tile 1710b. The circumferential bar 1760 may have a residual filled section 1780 to indicate the Max value for the corresponding candidate SEC, as a proportion of the maximum stimulus intensity. When stimulation at a candidate SEC is turned on, an element 1790 may be animated within the circumferential bar 1760, such as by traversing around the circumferential bar 1760, to dynamically indicate the progress of the stimulus intensity during the ramp towards the comfortable stimulus intensity for that SEC. The position of the element 1790 within the circumferential bar 1760 is indicative of the current stimulus intensity as a proportion of the maximum stimulus intensity. The current stimulus intensity in relation to the Max value, which is indicated by the residual filled section 1780, may therefore be easily perceived by a user.

The Next control 1740, illustrated in FIG. 17b, is enabled after at least one toggle switch has been activated. In some implementations, an additional criterion for enabling the Next control 1740 is that stimulation according to the final selected coverage needs to have been active for a minimum duration, for example five seconds. Once the patient activates the Next control 1740, the APM records the currently activated candidate SECs as the selected SECs, and stimulation is stopped on all SECs.

In an alternative implementation of the coverage selection stage 1070, there are no toggle switches, only tiles. In such an implementation, the Next control 1740 is enabled after at least one tile has been activated. Once the patient activates the Next control 1740, the APM records the currently activated candidate SECs as the selected SECs, and stimulation is stopped on all SECs.

If the patient selected no SECs before activating the Next control 1740, the workflow 1050 ends unsuccessfully.

In another implementation of the coverage selection stage 1070, there are no tiles or switches. Instead, the coverage selection screen 1700 displays a list of all possible combinations of the candidate SECs. For example, in an implementation in which there are four successful candidate SECs, the coverage selection screen displays the fifteen (15) possible combinations of the successful candidate SECs, e.g. “A” alone, “B” alone, “C” alone, “D” alone, “A” and “B” together, “A” and “C” together, etc. The patient may select any one of the combinations in the list, which turns on stimulation at all of the SECs in the selected combination at their respective comfortable stimulus intensities (and turns off stimulation at any previously selected combination). The selected SECs at the end of the coverage selection stage 1070 according to this implementation are those SECs in the selected combination when the Next control 1740 is activated.

The coverage selection stage 1070 is then configured to choose a primary SEC/MEC combination from among the selected SECs based on analysis of the data gathered during the PCSR stage 1060, and calculate therapy parameters for the primary SEC/MEC combination. This analysis is described below.

The primary SEC in the determined program is the selected SEC from which neural responses are measured to drive the feedback loop to adjust the stimulus intensity of the all SECs in accordance with the system 900b as described above in relation to FIG. 9b. Neural responses evoked by the non-primary selected SECs are not recorded or analysed. The stimulus intensities of the SECs are adjusted by the controller 116 so they remain in fixed ratios with the stimulus intensity of the largest SEC as described above in relation to FIG. 9b. The ratios to which the selected SECs are fixed may be saved in the determined program as the ratios of their respective comfortable stimulus intensities to the comfortable stimulus intensity of the largest SEC.

### Battery Single-Charge Life Estimation

Programs with multiple SECs, such as those produced by the APS 1000, potentially consume more power than programs with only one SEC. Single-charge life (SCL) for a rechargeable battery is therefore generally shorter for such programs than for single-SEC programs, possibly unacceptably shorter. It is therefore advantageous for the APS 1000 to estimate pre-emptively, that is, before the program is finalised, the SCL of the device 710 from the SECs being selected at the coverage selection stage 1070, at their respective comfortable stimulus intensities. If the estimated SCL is shorter than some predetermined limit, an alert may be raised to the patient to reconsider their selected SECs.

FIG. 17c is an illustration of a coverage selection screen 1770, which is similar to the coverage selection screen 1700. The new element is the SCL alert indicator 1785, which is displayed adjacent the Next control 1740 if the estimated SCL is below a predetermined threshold. The display of the SCL alert indicator 1785 is one example of the raising of an alert to a user according to the present technology.

In one implementation according to the present technology, the SCL (in hours) may be estimated using the following equation:

$$S C L = \frac{C ( 0 ) - C ( {V}_{min} )}{{f}_{s} \cdot ( m \cdot {αΣ}_{i = 1}^{N} ( {s}_{c omf , i} \cdot 2 \cdot {T}_{pw , i} ) + c ) + b} \qquad ( 9 )$$

The parameters of Equation (9) are as follows:
C(0) is the fully charged battery charge (in mA hours);
C(Vₘᵢₙ) is the minimal viable battery charge (in mA hours) i.e. the charge when the battery voltage reaches its minimum viable value Vₘᵢₙ;
fₛ is the stimulus frequency (in Hz)·
N is the number of selected SECs;
s_{comf,i} is the comfortable stimulus intensity (in mA) for the SEC numbered i (of N selected SECs);
T_{pw,j} is the pulse width for SEC numbered i (in secs);
α is a closed-loop factor;
m, b and c make up a linear model of actual charge consumption.

The parameters C(0) and C(Vₘᵢₙ) are available from the battery specifications.

The closed-loop factor α accounts for the variation of actual current delivered (due to the operation of the feedback loop as posture changes) from the comfortable stimulus intensity of each SEC. A value for α may be derived by finding a ratio between average delivered current during CLNS therapy and the comfortable stimulus intensity for a CLNS system. In an open-loop mode, the value of a would be 1.

The parameters m, b and c make up a linear model of actual charge consumption (per unit time) based on the estimated delivered current of the N SECs. The parameter m is a factor that scales delivered current to consumed current and accounts for the inefficiencies of the pulse generator 224. A value of m for a device 100 may be derived by estimating a ratio of current consumed by the device to delivered current.

Similarly, the parameter c is a term (in mC) that represents an overhead charge consumed by the electronics module 110 in each stimulus cycle. The overhead charge is charge that is not dependent on delivered current, such as current drawn by the measurement circuitry 128 and by the electrode selection module 126 in each stimulus cycle. A value of c for a device 100 may be estimated by measuring the average charge consumed by the electronics module 110 in each stimulus cycle that is not dependent on delivered current.

The parameter b represents a quiescent current (in mA) that is consumed by the module 110 independently of the stimulus and measurement parameters. A value of b for a device 100 may be estimated by measuring the average quiescent current (in mA) that is consumed by the device independently of the stimulus and measurement parameters.

The denominator of equation (9) therefore represents the charge consumption per unit time of the device 100 with N selected SECs operating at their respective comfortable stimulus intensities.

The denominator of equation (9) could optionally be further scaled by a usage fraction U representing the fraction of elapsed time that the device 100 is expected to be used (recalling that the patient has the ability to turn stimulation on and off using the remote controller 720). The usage fraction U could be derived from population data of device usage, or from past usage by the patient of the device 100, or some combination of the two.

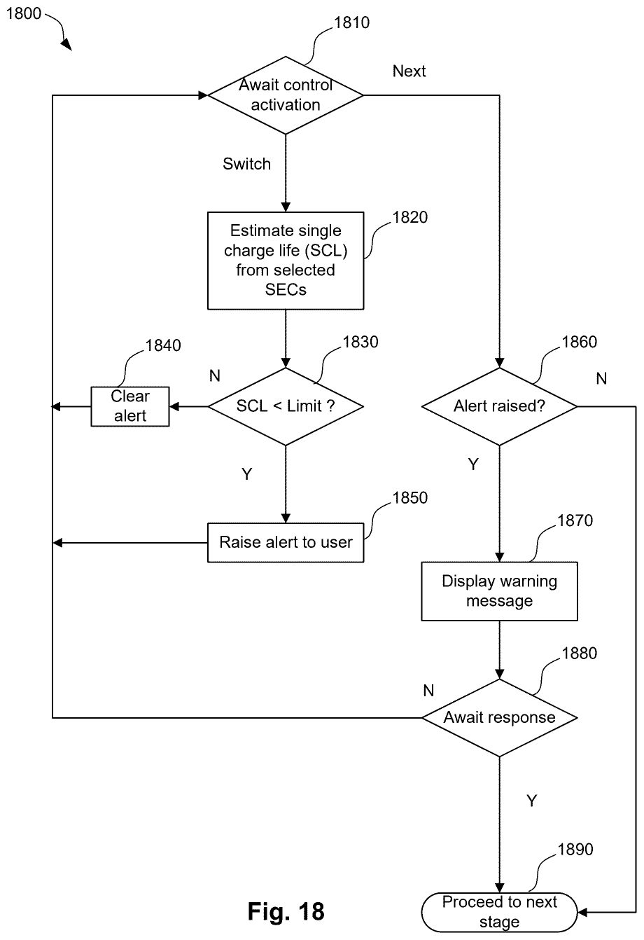

FIG. 18 is a flow chart illustrating a method 1800 of pre-emptively estimating the SCL for the SECs selected during the coverage selection stage 1070 and acting on that estimate, according to an aspect of the present technology. The method 1800 may be carried out by the APM and the device 710 according to one implementation of the coverage selection stage 1070. The method 1800 starts at step 1810 which awaits the activation of a control on the coverage selection screen 1700. If a switch control, e.g. 1720b, is activated (“Switch”), either to turn on or turn off stimulation at the corresponding SEC, a change to the selected SECs has taken place. (Note the method 1800 omits the activation of tile controls, which does not implicate SCL estimation.) The method 1800 then proceeds to step 1820, at which the APM estimates the SCL based on the currently selected SECs and their respective comfortable stimulus intensities. Step 1820 may use Equation (9) to estimate the SCL. Step 1830 then checks whether the estimated SCL is less than a predetermined limit, equal to 3 days (72 hours) in one implementation. If so (“Y”), step 1850 raises an alert to the user. In one example, the raising of an alert comprises displaying an SCL alert indicator 1785 adjacent the Next control 1740 of the coverage selection screen 1770, as illustrated in FIG. 17c. If not (“N”), step 1840 clears any previously raised alert, and the method 1800 returns to step 1810 to await activation of a control.

If at step 1810 the Next control 1740 is activated (“Next”), indicating the user has finished selecting SECs, the method 1800 proceeds to step 1860, which checks whether an alert is currently raised, indicating the SCL estimate for the selected SECs exceeds the predetermined limit. If not (“N”), the method 1800 at step 1890 proceeds to the next stage with the selected SECs. If so (“Y”), step 1870 displays a warning message and asks the user to confirm they wish to proceed. In one implementation, the message comprises the text “Please consult your clinician. By selecting this combination, the battery life of your device may be” [SCL estimate from step 1820] “vs” [predetermined limit from step 1830] “recommended for most patients. You may need to recharge your device more frequently.” The method 1800 then awaits the user's response at step 1880. If the user wishes to proceed (“Y”), the method 1800 proceeds to the next stage with the selected SECs (step 1890). If not (“N”), the method 1800 returns to step 1810 to await activation of a control on the coverage selection screen 1700.

Another implementation of SCL estimation during coverage selection is suitable for the “display list” implementation of the coverage selection stage 1070 described above. According to this implementation, the SCL is estimated for each of the combinations of SECs in the list to be displayed. If the estimated SCL for a combination exceeds the predetermined limit, that combination is either excluded from the list or displayed with a warning indicator indicating its potential for more frequent recharging. If such a combination is selected, a warning message may be displayed and confirmation to proceed sought as described above in relation to steps 1870 and 1880.

Yet another implementation of SCL estimation during coverage selection is a hybrid of the two implementations already described. Once an SEC is selected using a switch control on the coverage selection screen 1700, all combinations involving the selected SEC are enumerated and their respective SCLs estimated. Those combinations with an SCL that exceeds the predetermined limit are not permitted to be selected, or may only be selected after a confirmation to proceed.

In a further alternative implementation of SCL estimation during coverage selection, a quantitative indicator is displayed on the coverage selection screen in addition to, or instead of, the SCL alert indicator 1785. In such an implementation, the quantitative indicator is configured to represent the estimate of SCL obtained at step 1820. In one example, the quantitative indicator is the estimate itself in numeric form. In another example, the quantitative indicator is a concise representation such as a red-amber-green “traffic light” indicator which is red if the SCL estimate is less than the limit of step 1830, amber if the SCL estimate is above the limit of step 1830 but below a higher “caution” limit (e.g. twice the limit of step 1830), and green otherwise.

In a further alternative implementation of SCL estimation during coverage selection, rather than raising an alert as in step 1850, a mitigation action is taken that would increase the SCL, such as decreasing the stimulus frequency of the program or the pulse width of one or more of the stimsets. This may be accompanied by the display of a quantitative indicator as previously described so the patient may try out the effect of the mitigation while being informed of its effect on the SCL. The patient is then offered the option to accept or reflect the mitigation before proceeding with coverage selection.

### Data Analysis During the Coverage Selection Stage

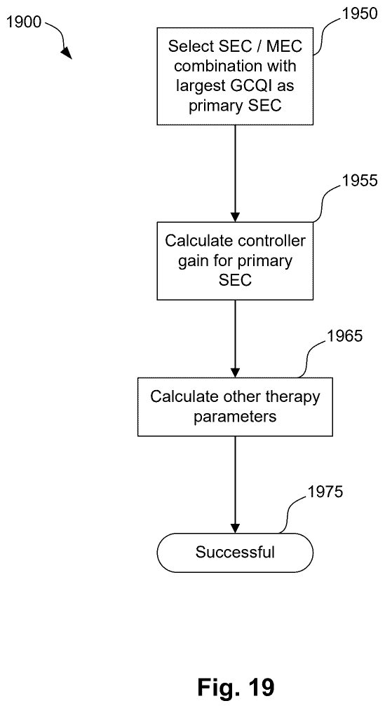

FIG. 19 contains a flowchart illustrating a data analysis method 1900 carried out by the APM and the device 710 during or after the coverage selection stage 1070 according to one implementation of the APM. The method 1900 starts at step 1950, which selects the SEC/MEC combination with the largest GCQI as the primary SEC for the program.

Step 1955 then calculates the gain K of the gain element 336 of the system 900b from the patient sensitivity S of the activation plot corresponding to the primary SEC. In one implementation, step 1955 calculates the gain K as

$$K = \frac{cos ω - 1 + \sqrt{c o {s}^{2} ω - 4 cos ω + 3}}{s} \qquad ( 10 )$$

where

$$ω = \frac{2 π {f}_{c}}{{f}_{s}} ,$$

f_{c} is a loop cutoff frequency, and fₛ is the stimulus frequency. In one implementation, the loop cutoff frequency is set to 3 Hz to balance the attenuation of noise with the attenuation of postural disturbances such as heartbeat.

Step 1965 calculates other therapy parameters for the multi-stimset CLNS system 900. In one implementation, the therapy parameters are:
A target ECAP amplitude d_{tgt}. This parameter may be calculated using equation (8) as the value of ECAP amplitude d on the fitted activation plot corresponding to the comfortable stimulus intensity s_{comf}, i.e. d_{tgt}=d(s_{comf}).
A maximum therapeutic stimulus intensity (not to be confused with the maximum stimulus intensity). This may be set to the Max value sₘₐₓ for the primary SEC.
A maximum target ECAP amplitude d_{tgt}(max). This may be set to the value of ECAP amplitude d on the fitted activation plot corresponding to the Max value sₘₐₓ for the primary SEC, i.e. d_{tgt(max)}=d(sₘₐₓ).
A minimum target ECAP amplitude d_{tgt(min)}. This may be set to zero.

At step 1975, the coverage selection stage 1070 ends, and the workflow 1050 is deemed successful.

In an alternative implementation of the coverage selection stage 1070, there is no primary SEC. Instead, each selected SEC runs its own independent feedback loop via its corresponding MEC, assuming an MEC of sufficient quality for each selected SEC has been found at the PCSR stage. In such an implementation, a modified method 1900 is carried out. The modified method 1900 has no step 1950. Instead, steps 1955 and 1965 are carried out for each selected SEC. At step 1975, the coverage selection stage 1070 ends, and the workflow 1050 is deemed successful.

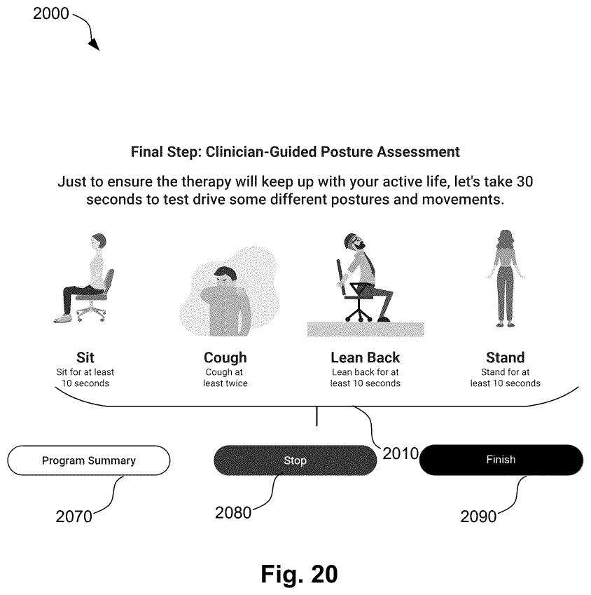

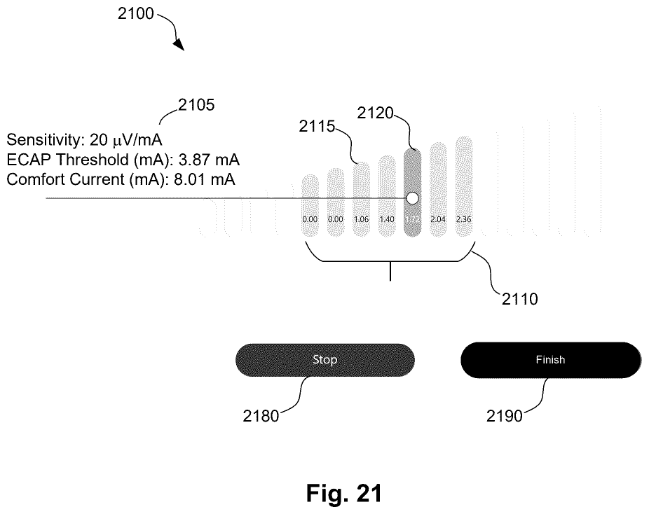

If the workflow 1050 completes successfully, the APM displays a posture assessment screen. FIG. 20 illustrates a posture assessment screen 2000 according to one implementation of the present technology. The posture assessment screen prompts the patient to test some different postures and movements, such as sitting, standing, leaning back while sitting, and coughing, illustrated by respective FIGS. 2010, while stimulation according to their final therapy program is active. The posture assessment screen also contains a Stop control 2080, activation of which stops all stimulation. The posture assessment screen also contains a Finish control 2090. When the Finish control 2090 is activated, the APM loads the therapy program to the device 710 and ends.

In some implementations, the posture assessment screen also contains a Program Summary control 2070. Activation of the Program Summary control 2070 brings up a Program Summary screen. FIG. 21 illustrates a Program Summary screen 2100 according to one implementation of the present technology. The Program Summary screen comprises some textual information 2105 about the final therapy program, such as the sensitivity S, the ECAP threshold s_{T}, and the comfortable stimulus intensity s_{comf} of the primary SEC. In some implementations, the textual information 2105 may also comprise an estimate of the SCL as obtained during the coverage selection stage 1070 using the method 1800 described above.

The Program Summary screen 2100 may also contain a stack 2110 of bars, e.g. the bar 2115. The stack 2110 is intended to replicate a stack of bars that is displayed on the remote controller 720. Each bar in the stack 2110 (illustrated as a horizontal stack in the screen 2100) represents a sub-range of target ECAP amplitude values d_{tgt} within the overall range [d_{tgt(min)}, d_{tgt(max)}] of target ECAP amplitude values. The sub-range represented by the bar numbered n may be determined as follows:

$${d}_{tgt ( min )} + [ ( n - 1 ) \frac{{d}_{tgt ( max )} - {d}_{tgt ( min )}}{{N}_{b}} , n \frac{{d}_{tgt ( max )} - {d}_{tgt ( min )}}{{N}_{b}} ] \qquad ( 11 )$$

where n=1, . . . , N_{b} and N_{b} is the number of bars in the stack 2110.

The bar that represents the sub-range that includes the target ECAP amplitude d_{tgt} calculated at step 1965 as part of the therapy parameters may be rendered differently from the other bars. For example, in the screen 2100, the bar 2120 represents the sub-range that includes the target ECAP amplitude d_{tgt}, and the bar 2120 is therefore rendered in a darker shade than the other bars such as the bar 2115. In one implementation, the number n (d_{tgt}) of the bar that represents the sub-range that includes the target ECAP amplitude d_{tgt} may be determined using the following formula:

$$n ( {d}_{tgt} ) = Minimum ( {N}_{b} , 1 + Trunc ( \frac{( {N}_{b} - 1 ) * ( {d}_{t g t} - {d}_{tgt ( min )} )}{{d}_{tgt ( max )} - {d}_{tgt ( min )}} ) ) \qquad ( 12 )$$

In some implementations of the Program Summary Screen, such as the screen 2100, each bar is also labelled with a number. The labelled number for a bar is a value of dose ratio corresponding to the sub-range of target ECAP amplitudes the bar represents. The dose ratio value Dₙ corresponding to the n-th bar (for n=1, . . . , N_{b}) may be obtained as the stimulus intensity sₙ corresponding to the target value d_{tgt(n)} in the centre of the sub-range of target ECAP amplitude values represented by the n-th bar, divided by the ECAP threshold s_{T} for the primary SEC. That is,

$${D}_{n} = \frac{{s}_{n}}{{s}_{T}} \qquad ( 13 )$$

where

$$d ( {s}_{n} ) = {d}_{tgt ( n )} \qquad ( 14 )$$

according to the fitted activation plot for the primary SEC (e.g. equation (8)). For example, for the 5^{th} bar 2120 out of 7 bars illustrated in the screen 2100, the corresponding dose ratio value D₅ is 1.72, so the bar 2120 is labelled with 1.72.

The program summary screen 2100 also contains a Stop control 2180, activation of which stops all stimulation.

The program summary screen also contains a Finish control 2190. When the Finish control 2190 is activated, the APM loads the therapy program to the device 710 and ends.

Alternatively, the Program Summary screen 2100 may be displayed after the successful completion of the workflow 1050, i.e. before the posture assessment screen 2000. In such implementations, the Program Summary screen 2100 may have a control to activate the posture assessment screen 2100, but may lack a Finish control 2190. In such implementations, the posture assessment screen 2100 may lack a Program Summary control 2170. In such implementations, the Program Summary screen 2100 may include controls for manual adjustment of selected therapy parameters. The ordering of screens in such implementations ensures that the posture assessment is carried out on the manually adjusted therapy program.

If the workflow 1050 is unsuccessful, a final screen informs the patient that the assisted programming was unsuccessful, and that manual programming is required. Such a final screen contains a Finish control, activation of which ends the APM without loading a program to the device 710.

### ECAP-Only Workflow

As mentioned above, the ECAP-only workflow is an alternative to the workflow 1050 for implementing assisted programming. The ECAP-only workflow is predicated on a set of selected SECs, one of which has already been designated as the primary SEC. The ECAP-only workflow comprises a single iteration of the PCSR stage 1060 on the primary SEC. If the primary SEC is deemed unsuccessful, the ECAP-only workflow may end unsuccessfully. If the primary SEC is deemed successful, the result of the PCSR stage 1060 is a Max value, an MEC, a corresponding activation plot (defined by its parameters), and a GCQI value for the activation plot. The ECAP-only workflow then proceeds to carry out the steps 1955 and 1965 of the method 1900 on the primary SEC to determine the therapy parameters (the controller gain, the target ECAP amplitude, the maximum therapeutic stimulus intensity, and the maximum and minimum target ECAP amplitudes).

After the ECAP-only workflow ends, the APM displays the posture assessment screen 2100 (if the ECAP-only workflow was successful) or the final screen (if the ECAP-only workflow was unsuccessful) as described above for the workflow 1050.

### Interpretation

The technology disclosed herein may be implemented in hardware (e.g., using digital signal processors, application specific integrated circuits (ASICs) or field programmable gate arrays (FPGAs)), or in software (e.g., using instructions tangibly stored on non-transitory computer-readable media for causing a data processing system to perform the steps described herein), or in a combination of hardware and software. The disclosed technology can also be implemented as computer-readable code on a computer-readable medium. The computer-readable medium can include any data storage device that can store data which can thereafter be read by a computer system. Examples of the computer-readable medium include read-only memory (“ROM”), random-access memory (“RAM”), magnetic tape, optical data storage devices, flash storage devices, or any other suitable storage devices. The computer-readable medium can also be distributed over network-coupled computer systems so that the computer-readable code is stored or executed in a distributed fashion. The present technology is not limited to any particular programming language or operating system.

### Wireless

In the context of the present disclosure, the term “wireless” and its derivatives may be used to describe circuits, devices, systems, methods, techniques, communications channels, etc., that may communicate data through the use of modulated electromagnetic radiation through a non-solid medium. The term does not imply that the associated devices do not contain any wires, although in some embodiments they might not. In the context of the present disclosure, the term “wired” and its derivatives may be used to describe circuits, devices, systems, methods, techniques, communications channels, etc., that may communicate data through the use of modulated signals propagating through a conductive medium. The term does not imply that the associated devices are coupled by electrically conductive wires.

Wireless communication standards that can be accommodated include IEEE 802.11 wireless LANs and links, Bluetooth, and wireless Ethernet. The technology disclosed herein may be implemented using devices conforming to other network standards and for other applications, including, for example other WLAN standards and other wireless standards such as MICS.

### Processes

Unless specifically stated otherwise, as apparent from the following discussions, it is appreciated that throughout the specification discussions utilizing terms such as “processing”, “computing”, “comparing”, “estimating”, “calculating”, “determining”, “analysing” or the like, refer to the action or processes of a computer or computing system, or similar electronic computing device, that manipulate or transform data represented as physical, such as electronic, quantities into other data similarly represented as physical quantities, or to otherwise execute a predefined procedure suitable to effect the described actions.

### Processor

In a similar manner, the term “processor” may refer to any device or portion of a device that processes electronic data, e.g., from registers or memory, to transform that electronic data into other electronic data that, e.g., may be stored in registers or memory. A “computer” or a “computing device” or a “computing machine” or a “computing platform” may include one or more processors.

The methods described herein are, in one embodiment, performable by one or more processors that accept computer-readable (also called machine-readable) code containing a set of instructions that when executed by one or more of the processors cause the one or more processors to carry out at least one of the methods described herein. Any processor capable of executing a set of instructions (sequential or otherwise) that specify actions to be taken are included within the meaning of the term “processor”. Thus, one example is a typical processing system that includes one or more processors. The processing system further may include a memory subsystem including main RAM or a static RAM, or ROM.

### Networked or Multiple Processors

In alternative embodiments, the one or more processors operate as respective standalone device(s) or may be connected, e.g., networked to other processor(s), in a networked deployment. The one or more processors may operate in the capacity of a server or a client machine in server-client network environment, or as a peer machine in a peer-to-peer or distributed network environment. The one or more processors may form a web appliance, a network router, switch or bridge, or any machine capable of executing a set of instructions (sequential or otherwise) that specify actions to be taken by that machine.

Note that while some diagram(s) only show(s) a single processor and a single memory that carries the computer-readable code, those in the art will understand that many of the components described above are included, but not explicitly shown or described in order not to obscure the inventive aspect. For example, while only a single machine may be illustrated, the term “machine” shall also be taken to include any collection of machines that individually or jointly execute a set (or multiple sets) of instructions to perform any one or more of the methods discussed herein.

### Additional Implementations

Thus, one implementation of each of the methods described herein is in the form of a computer-readable medium carrying a set of instructions, e.g., a computer program that are for execution on one or more processors. Thus, as will be appreciated by those skilled in the art, aspects of the present technology may be implemented as a method, an apparatus such as a special purpose apparatus, an apparatus such as a data processing system, or a computer-readable medium. The computer-readable medium carries computer-readable code including a set of instructions that when executed on one or more processors cause the processor or processors to implement a method. Accordingly, aspects of the present technology may take the form of a method, an entirely hardware implementation, an entirely software implementation or an implementation combining software and hardware aspects. Furthermore, the present technology may take the form of a carrier medium (e.g., a computer program product) carrying computer-readable program code embodied in the medium.

### Carrier Medium

The software may further be transmitted or received over a network via a network interface device. While the carrier medium is shown in an example embodiment to be a single medium, the term “carrier medium” should be taken to include a single medium or multiple media (e.g., a centralized or distributed database, or associated caches and servers) that store the one or more sets of instructions. A carrier medium may take many forms, including but not limited to, non-volatile media, volatile media, and transmission media.

### Means For Carrying out a Method or Function

Furthermore, some of the implementations are described herein as a method or combination of elements of a method that can be implemented by a processor of a processor device, computer system, or by other means of carrying out the function. Thus, a processor with the necessary instructions for carrying out such a method or element of a method forms a means for carrying out the method or element of a method. Furthermore, an element described herein of an apparatus is an example of a means for carrying out the function performed by the element.

Those of skill would further appreciate that the various illustrative logical blocks, modules, and algorithm steps described in connection with the implementations disclosed herein may be implemented as electronic hardware, computer software running on a special purpose machine that is programmed to carry out the operations described in the present disclosure, or combinations of both. To clearly illustrate this interchangeability of hardware and software, various illustrative components, blocks, modules, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the exemplary implementations.

### Implementations

Reference throughout the present disclosure to “one implementation” or “an implementation” means that a particular feature, structure or characteristic described in connection with the implementation is included in at least one implementation of the present technology. Thus, appearances of the phrases “in one implementation” or “in an implementation” in various places throughout the present disclosure are not necessarily all referring to the same implementation, but may refer to different implementations. Furthermore, the particular features, structures or characteristics may be combined in any suitable manner, as would be apparent to one of ordinary skill in the art from this disclosure, in one or more implementations.

Similarly, it should be appreciated that in the above description of example implementations of the present technology, various features are sometimes grouped together in a single implementation, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects may lie in less than all features of a single foregoing disclosed implementation. Thus, the claims following the Detailed Description of the Present Technology are hereby expressly incorporated into this Detailed Description of the Present Technology, with each claim standing on its own as a separate implementation of the present technology.

Furthermore, while some implementations described herein include some, but not other features included in other implementations, combinations of features of different implementations are meant to be within the scope of the present technology, and form different implementations of the present technology, as would be understood by those in the art. For example, in the following claims, any of the claimed implementations can generally be used in any combination.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude or position to indicate that the value or position described is within a reasonable expected range of values or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that each value between two particular values is also disclosed. For example, if 10 and 15 are disclosed, then 11, 12, 13, and 14 are also disclosed.

### Different Instances of Objects

As used herein, unless otherwise specified the use of the ordinal adjectives “first”, “second”, “third”, etc., to describe a common object, merely indicates that different instances of like objects are being referred to, and is not intended to imply that the objects so described must be in a given sequence, either temporally, spatially, in ranking, or in any other manner.

### Specific Details

In the description provided herein, numerous specific details are set forth. However, it is understood that implementations of the present technology may be practiced without these specific details. In other instances, well-known methods, structures and techniques have not been shown in detail in order not to obscure an understanding of the present technology.

### Terminology

Throughout the present disclosure, the terms “a” and “an” mean “one or more”, unless expressly specified otherwise.

Throughout the present disclosure, the word “comprise”, or variations such as “comprises” or “comprising”, will be understood to imply the inclusion of a stated element, integer, or step, or group of elements, integers, or steps, but not the exclusion of any other element, integer, or step, or group of elements, integers, or steps.

Throughout the present disclosure, a statement that an element may be “at least one of” or “one or more of” a list of options is to be understood to mean that the element may be any one of the listed options, or may be any combination of two or more of the listed options.

Throughout the present disclosure, the word “or” is to be read inclusively rather than exclusively, except where otherwise indicated.

Neither the title nor any abstract of the present disclosure should be taken as limiting in any way the scope of the claimed invention.

Where the preamble of a claim recites a purpose, benefit or possible use of the claimed invention, it does not necessarily limit the claimed invention to having only that purpose, benefit or possible use.

In the present specification, terms such as “part”, “component”, “means”, “section”, or “segment” may refer to singular or plural items and are terms intended to refer to a set of properties, functions, or characteristics performed by one or more items having one or more parts. It is envisaged that where a “part”, “component”, “means”, “section”, “segment”, or similar term is described as consisting of a single item, then a functionally equivalent object consisting of multiple items is considered to fall within the scope of the term; and similarly, where a “part”, “component”, “means”, “section”, “segment”, or similar term is described as consisting of multiple items, a functionally equivalent object consisting of a single item is considered to fall within the scope of the term. The intended interpretation of such terms described in this paragraph should apply unless the contrary is expressly stated or the context requires otherwise.

The term “connected” or a similar term, should not be interpreted as being limited to direct connections only. Thus, the scope of the expression “an item A connected to an item B” should not be limited to items or systems wherein an output of item A is directly connected to an input of item B. It means that there exists a path between an output of A and an input of B which may be a path including other items or means. “Connected”, or a similar term, may mean either that two or more elements are in direct physical or causal contact, or that two or more elements are not in direct contact with each other yet still co-operate or interact with each other.

It will be appreciated by persons skilled in the art that numerous variations or modifications may be made to the present technology as shown in the specific implementations without departing from the spirit or scope of the invention as broadly described. For example, any formulas given above are merely representative of procedures that may be used. Functionality may be added or deleted from the block diagrams and operations may be interchanged among functional blocks. Steps may be added or deleted to methods described within the scope of the present technology. The disclosed implementations are, therefore, to be considered in all respects as illustrative and not limiting or restrictive.

The features described in relation to one or more aspects of the present technology are to be understood as applicable to other aspects of the present technology. More generally, combinations of the steps in the method(s) of the present technology or the features of the system(s) or device(s) of the present technology described elsewhere in the present disclosure, including in the claims, are to be understood as falling within the scope of the present disclosure.

### INDUSTRIAL APPLICABILITY

It is apparent from the above that the arrangements described are applicable to the health care industries.

### LABEL LIST

device 100
patient 108
electronics module 110
battery 112
telemetry module 114
controller 116
memory 118
clinical data 120
clinical settings 121
control programs 122
pulse generator 124
electrode selection module 126
measurement circuitry 128
ground 130
array 150
biphasic stimulus pulse 160
ECAPS 170
target fibres 180
communications channel 190
external computing device 192
pulse generator 224
CLNS system 300
clinical settings controller 302
target ECAP controller 304
box 308
box 309
controller 310
box 311
pulse generator 312
pulse generator 312A
pulse generator 312B
pulse generator 312C
pulse generator 312D
ECAP detector 320
ECAP detector 320D
summing element 313
summing element 313D
measurement circuitry 318
measurement circuitry 318D
signal window 319
signal window 319A
signal window 319B
signal window 319C
signal window 319D
ECAP detector 320
comparator 324
gain element 336
integrator 338
activation plot 402
ECAP threshold 404
discomfort threshold 408
perception threshold 410
therapeutic range 412
activation plot 502
activation plot 504
activation plot 506
ECAP threshold 508
ECAP threshold 510
ECAP threshold 512
target ECAP amplitude 520
ECAP 600
neural stimulation system 700
device 710
remote controller 720
CST 730
CI 740
charger 750
illustration 800
first stimulus pulse 810
ISI 815
stimulus pulse 820
stimulus pulse 830
stimulus pulse 840
stimulus pulse 850
ECAP 860
stimulus period 890
CLNS system 900a
CLNS system 900b
APS 1000
clinical interface 1005
processor 1010
instruction memory 1020
CPA 1030
APM 1040
APF 1045
workflow 1050
PCSR stage 1060
coverage survey stage 1065
coverage selection stage 1070
table 1100
graphical representation 1110
screen 1200
screen 1200b
screen 1200c
control 1210
instructions 1220
stop control 1230
next control 1240
progress bar 1250
circumferential bar 1260
animated filled section 1270
residual filled section 1280
residual filled section 1285
indicator 1288
analysis method 1300
step 1310
step 1320
step 1330
step 1340
step 1350
step 1355
step 1363
step 1368
step 1370
step 1380
step 1390
coverage survey screen 1400
coverage survey screen 1400b
stimulation control 1410
stimulation control 1410b
instructions 1420a
instructions 1420b
option 1430
next control 1440
progress bar 1450
circumferential bar 1460
indicium 1475
residual filled section 1480
element 1490
graph 1550
GGC model 1560
pair 1570
vertical line 1580
vertical line 1590
arrow 1595
coverage survey screen 1600
target current amplitude 1610
linear ramp profile 1620
instant 1630
interval 1640
interval 1650
threshold current amplitude 1660
coverage selection screen 1700
tile 1710a
tile 1710b
tile 1710c
tile 1710d
indicium 1715
switch 1720a
switch 1720b
switch 1720c
switch 1720d
stop control 1730
next control 1740
progress bar 1750
circumferential bar 1760
coverage selection screen 1770
residual filled section 1780
SCL alert indicator 1785
element 1790
method 1800
step 1810
step 1820
step 1830
step 1850
step 1860
step 1870
step 1880
step 1890
method 1900
step 1950
step 1955
step 1965
step 1975
posture assessment screen 2000
figures 2010
program summary control 2070
stop control 2080
finish control 2090
program summary screen 2100
textual information 2105
stack 2110
bar 2115
bar 2120
program Summary control 2170
stop control 2180
finish control 2190

1. A neurostimulation system comprising:
a neurostimulation device for controllably delivering neural stimuli, the neurostimulation device comprising:
a stimulus source configured to deliver neural stimuli via selected electrodes of a plurality of implanted electrodes to a neural pathway of a patient; and
a control unit configured to control the stimulus source to deliver each neural stimulus according to a stimulus intensity parameter; and
an external computing device in communication with the neurostimulation device, the external computing device comprising:
a display, and
a processor configured to:
initialise the stimulus intensity parameter;
render a stimulation control on the display;
render a graphical element adjacent the stimulation control on the display;
ramp, on receiving an activation of the stimulation control by a user, a value of the stimulus intensity parameter, while instructing the control unit to control the stimulus source to deliver the neural stimuli according to the ramping value of the stimulus intensity parameter; and
cease, upon receiving a de-activation of the stimulation control,
ramping the value of the stimulus intensity parameter,
wherein the graphical element is configured to:
dynamically indicate the ramping value of the stimulus intensity parameter; and
indicate a discomfort threshold for the selected electrodes.

2. The neurostimulation system of claim 1, wherein the processor is further configured to ramp down, upon receiving the de-activation, the value of the stimulus intensity parameter, while instructing the control unit to control the stimulus source to deliver the neural stimuli according to the down-ramping value of the stimulus intensity parameter.

3. The neurostimulation system of claim 1, wherein the processor is further configured to instruct, upon receiving the de-activation, the stimulus source to cease delivering the neural stimuli.

4. The neurostimulation system of claim 1, wherein the graphical element comprises a partially filled section, wherein a ratio of the partially filled section to an area of the graphical element is maintained at a ratio of the stimulus intensity parameter value to a predetermined maximum value of the stimulus intensity parameter.

5. The neurostimulation system of claim 4, wherein the partially filled section indicates that the stimulus intensity parameter value has reached a neural response threshold.

6. The neurostimulation system of claim 5, wherein the partially filled section indicates that the stimulus intensity parameter value has reached a neural response threshold by changing the appearance of the partially filled section.

7. The neurostimulation system of claim 4, wherein the graphical element comprises a residual filled section, wherein a ratio of the residual filled section to an area of the graphical element is equal to a ratio of the discomfort threshold to a predetermined maximum value of the stimulus intensity parameter.

8. The neurostimulation system of claim 4, wherein the graphical element is further configured to indicate a target stimulus intensity parameter value that would achieve a target Normalised Dose Ratio for the selected electrodes.

9. The neurostimulation system of claim 8, wherein the graphical element comprises a second residual filled section, wherein a ratio of the second residual filled section to an area of the graphical element is equal to a ratio of the target stimulus intensity parameter value to a predetermined maximum value of the stimulus intensity parameter.

10. The neurostimulation system of claim 4, wherein the graphical element is further configured to indicate a neural response threshold.

11. The neurostimulation system of claim 10, wherein the graphical element comprises a further residual filled section, wherein a ratio of the further residual filled section to an area of the graphical element is equal to a ratio of the neural response threshold to a predetermined maximum value of the stimulus intensity parameter.

12. The neurostimulation system of claim 1, wherein the processor is further configured to record, upon receiving the de-activation, the value of the stimulus intensity parameter as the discomfort threshold for the selected electrodes.

13. The neurostimulation system of claim 12, wherein the processor is further configured to program, using the discomfort threshold, the neurostimulation device to deliver neural stimuli to the patient.

14. The neurostimulation system of claim 1, wherein the stimulation control is configured to:
become activated upon the user interacting with the stimulation control; and
remain activated as long as the user continues to interact with the stimulation control; and
become de-activated as soon as the user ceases to interact with the stimulation control.

15. The neurostimulation system of claim 1 wherein the ramping of the value of the stimulus intensity parameter follows a threshold ramp profile, the threshold ramp profile comprising a rapid step to a non-zero threshold current amplitude followed by a gradual increase in stimulus intensity.

16. An automated method of controlling a neurostimulation device to deliver neural stimuli using an external computing device in communication with the neurostimulation device, the method comprising:
rendering, by a processor of the external computing device, a stimulation control on a display of the external computing device;
rendering, by the processor, a graphical element adjacent the stimulation control on the display;
ramping, by the processor, on receiving an activation of the stimulation control by a user, a value of a stimulus intensity parameter, while instructing the neurostimulation device to deliver the neural stimuli according to the ramping value of the stimulus intensity parameter via selected electrodes of a plurality of implanted electrodes; and
ceasing, by the processor, upon receiving a de-activation of the stimulation control, to ramp the value of the stimulus intensity parameter,
wherein the graphical element is configured to:
dynamically indicate the ramping value of the stimulus intensity parameter; and
indicate a discomfort threshold for the selected electrodes.

17. The method of claim 16, further comprising ramping down, upon receiving the de-activation, the value of the stimulus intensity parameter, while instructing the neurostimulation device to deliver the neural stimuli according to the down-ramping value of the stimulus intensity parameter.

18. The method of claim 16, further comprising instructing, upon receiving the de-activation, the neurostimulation device to cease delivering the neural stimuli.

19. The method of claim 16, wherein the graphical element comprises a partially filled section, the method further comprising maintaining a ratio of the partially filled section to an area of the graphical element at a ratio of the stimulus intensity parameter value to a predetermined maximum value of the stimulus intensity parameter.

20. The method of claim 19, wherein the partially filled section indicates that the stimulus intensity parameter value has reached a neural response threshold.

21. The method of claim 20, wherein the partially filled section indicates that the stimulus intensity parameter value has reached a neural response threshold by changing the appearance of the partially filled section.

22. The method of claim 19, wherein the graphical element comprises a residual filled section, wherein a ratio of the residual filled section to an area of the graphical element is equal to a ratio of the discomfort threshold to a predetermined maximum value of the stimulus intensity parameter.

23. The method of claim 19, wherein the graphical element is further configured to indicate a target stimulus intensity parameter value that would achieve a target Normalised Dose Ratio for the selected electrodes.

24. The method of claim 23, wherein the graphical element comprises a second residual filled section, wherein a ratio of the second residual filled section to an area of the graphical element is equal to a ratio of the target stimulus intensity parameter value to a predetermined maximum value of the stimulus intensity parameter.

25. The method of claim 19, wherein the graphical element is further configured to indicate a neural response threshold.

26. The method of claim 25, wherein the graphical element comprises a further residual filled section, wherein a ratio of the further residual filled section to an area of the graphical element is equal to a ratio of the neural response threshold to a predetermined maximum value of the stimulus intensity parameter.

27. The method of claim 16, further comprising recording, upon receiving the de-activation, the value of the stimulus intensity parameter as the discomfort threshold for the selected electrodes.

28. The method of claim 27, further comprising programming, using the discomfort threshold, the neurostimulation device to deliver neural stimuli.

29. The method of claim 16, wherein the stimulation control is configured to:
become activated upon the user interacting with the stimulation control; and
remain activated as long as the user continues to interact with the stimulation control; and
become de-activated as soon as the user ceases to interact with the stimulation control.

30. The method of claim 16 wherein the ramping of the value of the stimulus intensity parameter follows a threshold ramp profile, the threshold ramp profile comprising a rapid step to a non-zero threshold current amplitude followed by a gradual increase in stimulus intensity.
